(19) United States
(12) Patent Application Publication
(10) Pub. No.: US 2026/0209690 A1
(43) Pub. Date: Jul. 23, 2026

(54) METHOD FOR TREATING CROSSLINKED MICROCOMPARTMENTS COMPRISING BIOLOGICAL ELEMENTS

(71) Applicant: TREEFROG THERAPEUTICS, PESSAC (FR)

(72) Inventors: Justine PLETENKA, PESSAC (FR); Elisa LUQUET, PESSAC (FR); Clément RIEU, PESSAC (FR); Pauline OLSEN, Cambridge, MA (US)

(21) Appl. No.: 19/448,383

(22) Filed: Jan. 14, 2026

(30) Foreign Application Priority Data
Jan. 22, 2025 (FR) FR2500659

(51) Int. Cl. C12N 5/00; C12N 5/074

(52) U.S. Cl. CPC C12N 5/0012; C12N 5/0696; C12N 2533/74; C12N 2537/10

(57) ABSTRACT

The present invention relates to the treatment of three-dimensional microcompartments comprising biological elements encapsulated in a crosslinked hydrogel. In particular, the invention relates to a method for treating a three-dimensional microcompartment comprising at least one biological element encapsulated in a crosslinked hydrogel, so as to reinforce the mechanical properties of said microcompartment, characterized in that it comprises a step of rinsing the microcompartment in a rinsing solution comprising at least one cation.

### TECHNICAL FIELD

The present invention relates to the treatment of three-dimensional microcompartments comprising biological elements encapsulated in a crosslinked hydrogel. In particular, the invention relates to a method for treating said microcompartments, a method for preparing said microcompartments, a microcompartment obtained by said preparation method, and a kit for implementing said method.

### BACKGROUND ART

The present invention falls within the field of microcompartments comprising biological elements, and their use in various industrial applications. Microcompartments, three-dimensional, partially or totally enclosed structures comprising one or more biological elements, play a crucial role in many biological and biotechnological processes.

These three-dimensional structures enable the encapsulation and cultivation of various biological elements in a controlled environment, thus opening up new prospects for cell therapy, tissue engineering and the production of molecules of interest.

However, a major challenge facing the large-scale use of microcompartments is their fragility in the face of mechanical stress, particularly due to changes in their properties caused by their storage environment (for example culture media). Indeed, these structures are susceptible to damage, which can lead to their breakage, during routine handling operations such as pipetting, centrifuging or filtration. This sensitivity to mechanical stress considerably limits their potential use in industrial contexts, where robustness and reproducibility are essential.

As a result, there is a need to develop solutions that improve the mechanical strength of microcompartments while preserving their functional properties and notably the viability of encapsulated biological elements.

There is therefore a need for an innovative solution to improve the mechanical strength of three-dimensional microcompartments comprising biological elements encapsulated in a crosslinked hydrogel, without compromising their essential functional properties such as the viability of the encapsulated biological elements. This solution should be applicable on a large scale, economically viable, and compatible with existing industrial processes, in order to unlock the full potential of microcompartments in various biotechnological and industrial applications.

### SUMMARY OF THE INVENTION

To meet this need, the invention offers a method for treating a three-dimensional microcompartment comprising at least one biological element encapsulated in a crosslinked hydrogel, so as to reinforce the mechanical properties of said microcompartment.

To achieve this, the method according to the invention comprises a step of rinsing said microcompartment in a rinsing solution comprising at least one cation.

Advantageously, this step of rinsing with a solution containing at least one cation reinforces the structure of the crosslinked hydrogel, thus improving the mechanical strength of the microcompartment. This allows the microcompartments to be handled and treated more efficiently without damaging them.

This approach solves the problem of the fragility of the microcompartments in the face of mechanical stress by strengthening the ionic bonds within the crosslinked hydrogel, which increases its structural stability and its resistance to mechanical stress.

The rinsing solution can be a solution in which at least one cation has a concentration of between 0.5 and 50 mM.

Advantageously, this concentration range enables effective reinforcement of the crosslinked hydrogel structure without compromising the viability of the encapsulated biological elements. Indeed, the cation concentration of the rinsing solution is high enough to improve the mechanical strength of the microcompartment, but low enough not to disturb the environment of the encapsulated biological elements.

At least one cation in the rinsing solution can be selected from calcium, iron, barium, strontium, copper, lead, aluminum, magnesium, manganese, zinc, cobalt, nickel ions and combinations thereof.

Advantageously, these cations are particularly effective in enhancing the mechanical properties of microcompartments via the interaction with the crosslinked hydrogel commonly used in the manufacture of microcompartments, such as alginate.

By using cations that interact strongly with the functional groups of the crosslinked hydrogel, optimal reinforcement of the structure is achieved without the need for high concentrations that could be harmful to biological elements. This reinforcement can be achieved by combining different improvements in mechanical properties, such as self-repair, malleability, elasticity, plasticity, deformability, resistance to punctiform stress, resistance to shear stress, resistance to compression, based on the cations used. Whatever the specific properties improved, the end result is a reduction, or even prevention, of damage to the microcompartments during mechanical stress, by virtue of the rinsing step according to the invention.

At least one cation in the rinsing solution may be a divalent or trivalent cation.

Advantageously, divalent and trivalent cations are particularly effective at creating strong ionic bonds within the crosslinked hydrogel, thus significantly reinforcing its structure.

According to one embodiment, the rinsing solution comprises calcium at a concentration of between 0.5 and 10 mM, preferentially between 2.5 and 5 mM.

Advantageously, calcium is a divalent cation particularly effective in reinforcing the structure of the crosslinked hydrogel, notably in the case of alginate-based hydrogels. Calcium forms ionic bonds with the alginate chains, which increases the rigidity and the mechanical strength of the microcompartment.

According to one embodiment, the rinsing solution comprises zinc at a concentration of between 0.1 and 5 mM, preferentially between 0.3 and 1 mM, even more preferentially between 0.4 and 0.6 mM.

Advantageously, zinc is a divalent cation that is particularly effective in enhancing the mechanical properties of microcompartments while preserving the viability and the proliferation capacity of encapsulated biological elements, notably pluripotent stem cells.

According to a particular embodiment, the rinsing solution comprises zinc sulfate (ZnSO₄).

According to another embodiment, the rinsing solution comprises copper at a concentration of between 0.1 and 5 mM, preferentially between 0.3 and 2 mM, even more preferentially between 0.5 and 1 mM.

Advantageously, copper helps reinforce the structure of the crosslinked hydrogel while maintaining conditions compatible with cell growth and maintenance of the pluripotency of the encapsulated cells.

According to a particular embodiment, the rinsing solution comprises copper chloride (CuCl₂).

According to one embodiment, the rinsing solution comprises calcium and zinc, preferentially calcium at a concentration of between 0.5 and 10 mM and zinc at a concentration of between 0.1 and 1 mM.

According to another embodiment, the rinsing solution comprises calcium and copper, preferentially calcium at a concentration of between 0.5 and 10 mM and copper at a concentration of between 0.1 and 2 mM.

Advantageously, these combinations of cations have a synergistic effect on enhancing the mechanical properties of the microcompartment.

According to one variant, the microcompartment may be hollow and comprise an outer hydrogel layer defining an inner part comprising at least one biological element.

Advantageously, this hollow structure provides a suitable space for the biological elements present within the inner part.

The microcompartment can be hollow, and consist of an outer hydrogel layer encapsulating a hollow inner part comprising at least one or more biological element(s), and optionally culture medium and/or extracellular matrix elements.

Advantageously, this configuration creates a controlled microenvironment for the biological elements.

This variant of the invention thus creates a favorable environment for biological elements while providing mechanical protection. Indeed, the hollow inner part can be filled with culture medium and extracellular matrix elements adapted to the specific needs of the biological elements, while the cation-reinforced outer layer ensures the protection and the integrity of the microcompartment.

According to another variant, the microcompartment can be a solid hydrogel ball encapsulating at least one biological element.

Advantageously, this configuration offers maximum protection for the encapsulated biological elements and enables a uniform distribution of cations throughout the volume of the microcompartment.

This variant of the invention thus makes it possible to protect biological elements in mechanically demanding environments. Indeed, the solid structure provides a complete physical barrier around biological elements, while the cation treatment improves the mechanical strength of the entire microcompartment.

The hydrogel constituting the microcompartment can be selected notably from alginates, carrageenans, pectins, gelatins, collagen, hyaluronic acid, chitosan and combinations thereof.

Advantageously, these hydrogels are biocompatible and offer good versatility, both in terms of their mechanical properties and their encapsulation capacity.

According to one embodiment, the hydrogel of the microcompartment has a shear modulus of between 30 and 80 kPa, preferentially between 40 and 70 kPa.

Advantageously, this shear modulus gives the microcompartment a mechanical strength suitable for industrial handling while maintaining sufficient flexibility to preserve the integrity of the encapsulated biological elements.

The Young's modulus E of the hydrogel can be expressed based on the shear modulus G according to the relationship E=2G(1+v), where v is Poisson's ratio. For an incompressible hydrogel where v=0.35, this relationship simplifies to E=3G.

According to one embodiment, the hydrogel has:
a shear modulus of between 30 and 80 kPa, preferentially between 40 and 70 kPa; and/or
a Young's modulus of between 100 and 250 kPa, preferentially between 100 and 200 kPa.

Thus, depending on the specific needs of the encapsulated biological elements, each type of hydrogel offers unique characteristics in terms of porosity, rigidity and interactions with the biological elements, making it possible to select the most suitable material for each application.

At least one biological element encapsulated in the microcompartment may notably be selected from the following biological elements: a cell, a cell organelle, a cell fragment, a cell aggregate, an organoid, DNA, RNA, a protein, a microorganism, a vector, a vesicle, an exosome, a microtissue, a spheroid, an embryoid body and combinations thereof.

Advantageously, this wide range of biological elements that can be encapsulated offers numerous possibilities for applications in the fields of regenerative medicine and tissue engineering.

The microcompartment can have various three-dimensional shapes, notably the shape of an ovoid, a cylinder, a spheroid, a sphere, a teardrop or a fiber. The microcompartment can be enclosed or partially enclosed.

Advantageously, these different shapes enable microcompartments to be adapted to various experimental or therapeutic applications and constraints.

The smallest dimension of the microcompartment can be between 1 μm and 1.5 mm, preferentially between 100 μm and 800 μm.

The largest dimension of the microcompartment can be between 1 μm and 1.5 mm, preferentially between 120 μm and 800 μm, preferentially between 200 μm and 600 μm, even more preferentially 200 μm and 500 μm, notably between 200 μm and 250 μm.

In the context of the invention, the largest dimension of the microcompartment is always greater than the smallest dimension of the microcompartment.

Advantageously, this range of sizes makes it possible to create microcompartments small enough to be handled and/or administered easily, yet large enough to contain a significant number of biological elements.

The method can also comprise a step of mechanically stressing said rinsed microcompartment, wherein the mechanical stressing can be achieved notably by filtration, agitation in liquid medium, centrifugation, suction or a combination thereof.

The mechanical stressing step can be carried out without damaging the microcompartments, by virtue of the reinforced mechanical properties obtained during the rinsing step with the cation-containing solution.

The invention can enable the rinsed microcompartments to be subjected to a variety of controlled mechanical stresses while preserving their structural integrity. Mechanical stress can have different functions depending on the context, such as changing the culture medium, filtration, sampling, flow through a restricted pipe, agitation in a bioreactor or centrifugation to group together the biological elements.

The mechanical stress step may comprise applying a centrifugal acceleration of at least 10 g to at least one cellular microcompartment rinsed in accordance with the invention.

Advantageously, this centrifugal acceleration enables encapsulated biological elements to be grouped together, significantly reducing the number of isolated biological elements.

The rinsing step and/or the mechanical stress step can be carried out using at least one device comprising a body (2) having at least a first port (5) and a second port (6) and a filtering membrane (3) extending along a plane P in the body (2), thus defining two compartments and able to prevent the passage of microcompartments of biological elements from one compartment to the other, the first port (5) being intended for the injection and withdrawal of the cellular microcompartments immersed in a rinsing solution and/or a solution A and the second port (6) being intended for the withdrawal and injection of solution A and/or the rinsing solution, characterized in that the injection into the first port (5) takes place in a direction D that is not substantially orthogonal to the plane P of the filtering membrane (3).

Advantageously, this device enables the rinsing and/or mechanical stress steps to be carried out efficiently, preferentially simultaneously, while minimizing the risks of damage to the microcompartments.

This approach solves the problem of the delicate handling of the microcompartments during the rinsing and mechanical stress steps by using an injection that is not orthogonal to the plane of the filtering membrane, creating a tangential flow that reduces the risk of clogging and damage to the microcompartments, while ensuring efficient rinsing and controlled mechanical stress.

The invention also offers a method for preparing a three-dimensional microcompartment comprising the implementation of the following steps:
a) Encapsulation of at least one biological element in a crosslinked hydrogel to form a three-dimensional microcompartment; and
b) Treatment of the microcompartment obtained in step a) by any one of the embodiments of the treatment method according to the invention.

Advantageously, this preparation method makes it possible to optimize the production of microcompartments comprising biological elements in a controlled and reproducible manner.

In this way, the preparation method according to the invention makes it possible to increase the production capacity of the microcompartments by increasing the mechanical stresses/demands on the microcompartments.

Encapsulation step a) may comprise a step of crosslinking the hydrogel using a crosslinking solution comprising at least one crosslinking agent.

Preferentially, at least one crosslinking agent is calcium.

Advantageously, this crosslinking step forms the initial structure of the hydrogel, which is then reinforced by the treatment with the cation-containing rinsing solution in step b) of microcompartment treatment.

Preferentially, at least one crosslinking agent is calcium and has a concentration in the crosslinking solution of between 0.5 and 300 mM, preferentially a concentration of between 25 and 150 mM.

Advantageously, this concentration range makes it possible to finely control the degree of crosslinking of the hydrogel and therefore its initial mechanical properties.

At least one crosslinking agent from step a) may be distinct from at least one cation from step b).

Advantageously, this distinction between the initial crosslinking agent and the cations used for reinforcement enables finer control of the properties of the microcompartment at each step of its preparation.

At least one crosslinking agent from step a) can be identical to at least one cation from step b).

Advantageously, using the same agent for initial crosslinking and subsequent reinforcement can simplify the method and ensure optimum compatibility between the two steps.

According to one embodiment, step a) may comprise the following steps:
1) forming a hollow hydrogel microcompartment encapsulating biological elements, using an encapsulation device, preferentially comprising a micro-fluidic and/or milli-fluidic device and solutions, said solutions being preferentially the following:
   a hydrogel solution;
   optionally an intermediate solution, preferentially isotonic; and
   a solution comprising biological elements, culture medium and, optionally, the extracellular matrix and/or extracellular matrix substitute;
2) crosslinking the hydrogel, preferentially using a crosslinking solution, such as a crosslinking bath.

Advantageously, this method can be used to form hollow microcompartments with a well-defined structure, comprising an outer layer of crosslinked hydrogel and a hollow inner part containing the biological elements.

This approach solves the problem of creating microcompartments with a complex and controlled architecture using a micro-fluidic device to form multi-layer structures in a single step, while collection in a crosslinking solution ensures rapid gelation of the outer layer, thus preserving the structure of the microcompartment.

The invention also relates to a three-dimensional cellular microcompartment obtained by any one of the embodiments of the preparation method according to the invention.

Advantageously, this three-dimensional cellular microcompartment benefits from all the features and advantages described previously, notably in terms of mechanical strength, biocompatibility and versatility of application.

The invention also proposes the use of the cellular micro-compartment obtained by any one of the embodiments of the preparation method according to the invention for cell culture, drug testing, regenerative medicine or tissue engineering.

Finally, the invention proposes a kit comprising at least one rinsing solution comprising at least one cation in which at least one cation has a concentration of between 0.5 and 50 mM, for implementing a method according to any one of the preceding claims.

Advantageously, this kit facilitates the implementation of the micro-compartment treatment method by providing a ready-to-use rinsing solution with an optimum concentration of cations.

### BRIEF DESCRIPTION OF THE FIGURES

FIG. 1 is a perspective view of an example embodiment of a device capable of performing a rinsing and/or mechanical stress step in the context of the invention.

FIG. 2 is a schematic view of an example embodiment of a device capable of performing a rinsing and/or mechanical stress step in the context of the invention,

FIG. 3 is a perspective view of an example embodiment of a device capable of performing a rinsing and/or mechanical stress step in the context of the invention,

FIG. 4 is a half-sectional view of FIG. 3 along the sectional plane AA,

FIG. 5 is a half-sectional view of another example embodiment of a device capable of performing a rinsing and/or mechanical stress step in the context of the invention, and

FIG. 6 A is a microscopic observation of the microcompartments washed with a non-calcium-supplemented solution, accompanied by a photograph of the cell sieve after rinsing.

FIG. 6 B is a microscopic observation of the microcompartments washed with calcium-supplemented solution (2 mM), accompanied by a photograph of the cell sieve after rinsing.

FIG. 7 is a set of photographs taken using the microscope of cellular microcompartments harvested after rinsing with a rinsing solution comprising different concentrations of calcium, accompanied by a photograph of the filter after rinsing.

FIG. 8 is an observation taken using the microscope of a microcompartment rinsed with a rinsing solution comprising different concentrations of calcium at different flow rates.

FIG. 9 is a series of video-microscopy photos of microcompartments comprising cells from cell line no. 1.

FIG. 10 is a series of video-microscopy photos of microcompartments comprising cells from cell line no. 2.

FIG. 11 is a series of video-microscopy photos of microcompartments comprising cells from cell line no. 3.

FIG. 12 is a graphical representation of the cell viability measurement for cells from different cell lines from microcompartments obtained after a rinsing step using a rinsing solution comprising iron.

FIG. 13 is a graphical representation of the 5-day amplification factor measurement for cells from different cell lines from microcompartments obtained after a rinsing step using a rinsing solution comprising iron.

FIG. 14 is a graphical representation of the pluripotency measurement for cells belonging to cell line no. 3 from microcompartments obtained after a rinsing step using a rinsing solution comprising iron.

FIG. 15 is a graphical representation of the pluripotency measurement of cells belonging to cell line no. 2 from microcompartments obtained after a rinsing step using a rinsing solution comprising iron.

FIG. 16 is a diagram showing the experimental protocol for washing cell capsules with different solutions containing zinc (ZnSO₄) or copper (CuCl₂) at different concentrations.

FIG. 17 is a series of microscopic observations of cellular microcompartments during culture (day 1 to day 5) after rinsing with different solutions: 3 mM CaCl₂ (control), 0.5 mM ZnSO₄, 0.5 mM CuCl₂ and 1 mM CuCl₂.

FIG. 18 is a graphical representation of lactate production expressed in mmol/mL of capsules over time for different rinsing conditions.

FIG. 19 is a graphical representation of the measurement of cell pluripotency, showing the expression of the markers OCT4, SOX2 and OCT4/SOX2 for the different rinsing conditions.

FIG. 20 is a graphical representation of rheological measurements showing the storage modulus and the normal force based on sample thickness, as well as a bar chart of the shear modulus for different washing conditions.

### DESCRIPTION OF THE INVENTION

### Definitions

For the purpose of the invention, “° centrifugal acceleration” refers to the acceleration that occurs when an object or a body is in circular motion around a central point or along a curved trajectory. This acceleration is directed outwardly, away from the center of rotation, and it results from the inertia of the moving object. The centrifugal force results from the mass of the object or body multiplied by this centrifugal acceleration. Thus, centrifugal acceleration is related to the acceleration acquired by a body or an object under the effect of a centrifugal force.

For the purpose of the invention, “alginate” refers to linear polysaccharides formed from P-D-mannuronate and a-L-guluronate, salts and derivatives thereof.

For the purpose of the invention, “differentiated” cells refers to cells that display a particular phenotype, as opposed to pluripotent stem cells that are undifferentiated or progenitor cells that are in the process of differentiation.

For the purpose of the invention, “micro-fluidic device” refers to any device or any combination of devices provided with one or more inlets and one or more outlets interconnected by a plurality of channels with a cross-section of the order of a hundred micrometers and capable of directing a flow of one or more fluids from the inlet(s) to the outlet(s).

For the purpose of the invention, “milli-fluidic device” refers to any device or any combination of devices provided with one or more inlets and one or more outlets interconnected by a plurality of channels with a cross-section of the order of a millimeter and capable of directing a flow of one or more fluids from the inlet(s) to the outlet(s).

For the purpose of the invention, “human cells” refers to human cells or immunologically humanized non-human mammalian cells. Even where not specified, the cells, the stem cells, the progenitor cells and the tissues according to the invention are constituted by or are obtained from human cells or from immunologically humanized non-human mammalian cells.

For the purpose of the invention, “progenitor cell” refers to a stem cell already engaged in cellular differentiation but not yet differentiated.

For the purpose of the invention, “pluripotent stem cell” or “pluripotent cell” refers to a cell which has the ability to form all the tissues present in the entire organism of origin, without however being able to form an entire organism as such. Human pluripotent stem cells may be referred to as hPSCs in the context of the present invention. In particular, these may be induced pluripotent stem cells (iPSC or hiPSC for human induced pluripotent stem cells), embryonic stem cells or MUSE cells (for “Multilineage-differentiating Stress Enduring”).

For the purpose of the invention, “induced pluripotent stem cell” refers to a pluripotent stem cell induced to pluripotency by genetic reprogramming of differentiated somatic cells. These cells are notably positive for pluripotency markers such as alkaline phosphatase staining and expression of NANOG, SOX2, OCT4 and SSEA4/5 proteins. Examples of methods for obtaining induced pluripotent stem cells are described in the articles by Yu et al. (Science 2007, 318 (5858): 1917-1920), Takahashi et al (Cell, 207, 131(5): 861-872) and Nakagawa et al (Nat Biotechnol, 2008, 26(1): 101-106).

For the purpose of the invention, “cell layer” or “cell bed” refers to several cells forming a layer or a bed that can be structured around a lumen; it may for example be a tissue or a cellular micro-tissue or a three-dimensionally grouped culture. The thickness of the cell layer can be variable. This layer is organized in three dimensions in the microcompartment.

The “Feret diameter” of a microcompartment refers to the distance “d” between two tangents to said microcompartment, these two tangents being parallel, such that the entire projection of said microcompartment is between these two parallel tangents.

For the purpose of the invention, “drop” also refers to a three-dimensional structure formed from at least one liquid solution comprising the constituents of a non-crosslinked hydrogel (polymerization precursors, non-crosslinked or partially crosslinked polymer chains, etc.), hydrogel precursor elements. The drop is also a transitional state between co-injection of the various components and the microcompartment according to the invention.

For the purpose of the invention, “microcompartment” or “capsule” also refers to a partially or totally enclosed three-dimensional structure. It is formed from a matrix of polymer chains, a hydrogel, for example alginate, containing one or more biological elements. The biological elements can be diverse. They may notably include cells. Encapsulated cells can take a variety of forms, notably single cells, cell aggregates, cellular microtissues, multicellular aggregates, cellular tissues, or any other configuration enabling cell encapsulation, containment or culture. The microcompartment can be designed to be filled with one or more biocompatible materials, such as alginate, thus forming a solid microcompartment such as for example a solid ball. Alternatively, the microcompartment can be hollow, forming an internal cavity in which biological elements can be contained or cultivated. The structure thus consists of a rigidified outer hydrogel layer and an inner part comprising at least one cell and a hydrogel layer or mesh suitable for the culture of biological elements, notably cell culture.

For the purpose of the invention, “largest dimension” of a microcompartment refers to the value of the largest Feret diameter of said microcompartment.

For the purpose of the invention, “smallest dimension” of a microcompartment or of a layer of cells refers to the value of the smallest Feret diameter of said microcompartment.

For the purpose of the invention, “tissue” or “biological tissue” refers to the common meaning of tissue in biology, i.e. the intermediate level of organization between the cell and the organ. A tissue is a set of similar cells of the same origin (usually from a common cell lineage, although they may originate from an association of distinct cell lineages), grouped together in a cluster, network or bundle (fiber). A tissue forms a functional whole, i.e. its cells work together to perform the same function. Biological tissues regenerate regularly and are joined together to form organs.

For the purpose of the invention, “reinforce the mechanical properties” refers to improving the capacities of a material to withstand mechanical stresses, such as enhancing the elastic modulus and/or enhancing rupture parameters.

For the purpose of the invention, “biological element” means any entity of biological origin or involved in biological processes, comprising, but not limited to, cells (for example stem cells, differentiated cells, immune cells), cellular components (organelles, membranes), vectors such as transduction agents (a viral particle, a viral pseudoparticle), biomolecules (proteins, peptides, enzymes, nucleic acids, lipids, carbohydrates), or else derived entities (exosomes, extracellular vesicles, lysates).

### Method for Treating a Microcompartment

The present invention relates to a method for treating a three-dimensional microcompartment comprising at least one biological element encapsulated in a crosslinked hydrogel so as to reinforce the mechanical properties of said microcompartment.

To this end, the treatment method according to the invention comprises a step of rinsing said microcompartment in a rinsing solution comprising at least one cation.

Advantageously, the rinsing step reinforces the structure of the crosslinked hydrogel by creating additional ionic bonds to improve the mechanical properties of the microcompartment.

According to one variant, the treatment method according to the invention comprises a step of rinsing said microcompartment in a rinsing solution comprising at least one cation.

According to another variant, the treatment method according to the invention comprises several steps of rinsing said microcompartment in a rinsing solution comprising at least one cation.

Preferentially, the treatment method according to the invention comprises at least one, at least two, at least three, at least four, or at least five steps of rinsing said microcompartment in a rinsing solution comprising at least one cation.

In the context of the invention, the rinsing step is preferentially carried out by fully immersing a microcompartment in the rinsing solution comprising at least one cation. This total immersion ensures uniform contact between the rinsing solution and the entire surface of the microcompartment.

According to one embodiment, the rinsing solution comprises at least one cation selected from calcium, iron, barium, strontium, copper, lead, aluminum, magnesium, manganese, zinc, cobalt, nickel ions and combinations thereof.

Preferentially, at least one cation in the rinsing solution is a divalent or trivalent cation.

According to a particular embodiment, the rinsing solution comprises at least one divalent cation and at least one trivalent cation.

According to a particular embodiment, the rinsing solution comprises at least 2, 3, 4, 5 distinct cations.

The rinsing solution can vary in terms of composition and concentration of cations.

According to one embodiment, the rinsing solution is a solution in which at least one cation has a concentration of between 0.5 and 50 mM, notably between 0.5 and 25 mM.

A cation concentration of less than 0.5 mM is not sufficient to provide satisfactory reinforcement of the mechanical properties of the microcompartment. On the other hand, a concentration greater than 50 mM may adversely affect the viability of encapsulated biological elements.

Advantageously, within the range of values between 0.5 and 50 mM, different concentrations can be used according to the specific needs based on the cations used.

The concentration of cations in the rinsing solution can be adjusted within the concentration range described based on the specific needs of the microcompartment and the biological elements contained therein. A higher concentration of cations may lead to greater reinforcement of the mechanical properties, while a lower concentration may be preferable to maintain the viability of certain sensitive biological elements.

Preferably, the rinsing solution comprises at least calcium. According to a particular embodiment, the rinsing solution comprises only one type of cation: calcium.

Calcium is a particularly advantageous cation as it reinforces the structure of the crosslinked hydrogel, notably in the case of alginate-based hydrogels. Calcium forms ionic bonds with the alginate chains, which increases the rigidity and the mechanical strength of the microcompartment.

Preferentially, the rinsing solution comprises at least one divalent calcium (Ca²⁺).

Particularly suitably, the rinsing solution comprises calcium at a concentration of between 0.5 and 10 mM, preferentially between 2.5 and 5 mM.

In particular, the rinsing solution can comprise calcium at a concentration of between 1 and 8 mM, preferentially between 1 and 6 mM, more preferentially between 2 and 6 mM, even more preferentially between 2.5 and 5 mM.

According to one embodiment, the rinsing solution comprises calcium in a concentration of between 0.5 and 2 mM, notably between 0.5 and 1 mM.

According to another embodiment, the rinsing solution comprises at least iron. According to a particular embodiment, the rinsing solution comprises only one type of cation: iron.

Particularly suitably, the rinsing solution comprises iron at a concentration of between 0.5 and 10 mM, preferentially between 0.5 and 5 mM.

According to one embodiment, the rinsing solution comprises a ferric cation (Fe²⁺) and/or a ferrous cation (Fe³⁺).

According to another embodiment, the rinsing solution comprises calcium and at least one cation selected from iron (preferentially Fe²⁺ and/or Fe³⁺), barium, strontium, copper, lead, aluminum, magnesium, manganese, zinc, cobalt, nickel ions and combinations thereof.

According to one embodiment, the rinsing solution comprises between 0.5 and 10 mM of calcium and at least one cation selected from iron (preferentially Fe²⁺ and/or Fe³⁺), barium, strontium, copper, lead, aluminum, magnesium, manganese, zinc, cobalt, nickel ions and combinations thereof at a concentration of between 0.5 and 50 mM, notably between 0.5 and 25 mM, preferentially between 0.5 and 10 mM, more preferentially between 0.5 and 5 mM.

According to one embodiment, the rinsing solution comprises calcium ions (preferentially Ca²⁺) and iron ions (preferentially Fe²⁺ and/or Fe³⁺).

Preferentially, in this embodiment, the rinsing solution comprises between 0.5 and 10 mM of calcium (preferentially Ca²⁺) and between 0.5 and 9 mM of iron (preferentially Fe²⁺ and/or Fe³⁺).

According to another embodiment, the rinsing solution comprises at least zinc. According to a particular embodiment, the rinsing solution comprises only one type of cation: zinc.

Particularly suitably, the rinsing solution comprises zinc at a concentration of between 0.1 and 5 mM, preferentially between 0.3 and 1 mM, even more preferentially between 0.4 and 0.6 mM.

According to one embodiment, the rinsing solution comprises zinc sulfate (ZnSO₄).

Zinc is a divalent cation (Zn²⁺) that interacts with alginate carboxylate groups to form ionic bonds reinforcing the structure of the crosslinked hydrogel.

Furthermore, zinc is a trace element naturally present in cell culture media, which makes it particularly compatible with the maintenance of cell viability.

According to another embodiment, the rinsing solution comprises at least copper. According to a particular embodiment, the rinsing solution comprises only one type of cation: copper.

Particularly suitably, the rinsing solution comprises copper at a concentration of between 0.1 and 5 mM, preferentially between 0.3 and 2 mM, even more preferentially between 0.5 and 1 mM.

According to one embodiment, the rinsing solution comprises copper chloride (CuCl₂).

Copper is a divalent cation (Cu²⁺) that forms ionic bonds with the alginate chains, thus contributing to the mechanical reinforcement of the microcompartment. At appropriate concentrations, copper helps maintain the cell viability and proliferation capacity of encapsulated cells.

According to one embodiment, the rinsing solution comprises calcium and zinc, preferentially calcium at a concentration of between 0.5 and 10 mM and zinc at a concentration of between 0.1 and 1 mM.

According to another embodiment, the rinsing solution comprises calcium and copper, preferentially calcium at a concentration of between 0.5 and 10 mM and copper at a concentration of between 0.1 and 2 mM.

Advantageously, the combined use of several divalent cations can enable optimized mechanical properties to be obtained based on the specific needs of the intended application.

According to one embodiment, the microcompartment obtained after the rinsing step has a Young's modulus of between 3 kPa and 1.2 MPa, preferentially 3 and 800 kPa, notably between 3 and 600 kPa, between 3 and 500 kPa, preferentially between 3 and 350 kPa, even more preferentially between 100 and 200 kPa.

According to one embodiment, the microcompartment resulting from the rinsing step has a rupture stress of between 10 and 500 kPa, notably between 200 and 500 kPa.

In the context of the invention, the rupture stress of the microcompartment can be measured using a compression test, a torsion test or an extension test.

According to one embodiment, the microcompartment after from the rinsing step has:
a Young's modulus between 3 kPa and 1.2 MPa, preferentially between 3 and 800 kPa, notably between 3 and 600 kPa, between 3 and 500 kPa, preferentially between 3 and 350 kPa; and/or
a rupture stress of between 10 and 500 kPa, notably between 200 and 500 kPa.

According to one embodiment, the microcompartment is hollow and comprises an outer hydrogel layer defining an inner part, said inner part comprising at least one biological element. This hollow structure makes it possible to encapsulate a large volume of biological elements while maintaining an outer hydrogel layer. The crosslinked outer hydrogel layer acts as a semi-permeable barrier, allowing the exchange of nutrients and metabolites with the external environment while protecting the encapsulated biological elements.

According to one embodiment, the microcompartment obtained after the rinsing step is hollow and comprises an outer hydrogel layer having a Young's modulus of between 3 kPa and 1.2 MPa, preferentially between 3 and 800 kPa, notably between 3 and 600 kPa, between 3 and 500 kPa, preferentially between 3 and 400 kPa, more preferentially between 3 and 300 kPa, and defining an inner part, said inner part comprising at least one biological element.

According to one variant, the microcompartment obtained after the rinsing step comprises:
an outer hydrogel layer, and
an inner part comprising at least:
   at least one biological element, and
   a hydrogel layer or mesh arranged between the outer layer and at least one biological element.

According to one embodiment, the microcompartment obtained after the rinsing step comprises:
an outer hydrogel layer, and
an inner part comprising at least:
   at least one biological element, and
   a hydrogel layer or mesh arranged between the outer layer and at least one biological element, said hydrogel layer or mesh having a Young's modulus of between 0.01 and 200 kPa, notably between 0.1 and 60 kPa, even more preferentially between 0.1 and 5 kPa.

According to one embodiment, the microcompartment obtained after the rinsing step comprises:
an outer hydrogel layer, said hydrogel having a Young's modulus of between 3 kPa and 1.2 MPa, preferentially between 3 and 800 kPa, notably between 3 and 600 kPa, 3 and 500 kPa, preferentially between 3 and 400 kPa, more preferentially between 3 and 300 kPa, and
an inner part comprising at least:
   at least one biological element, and
   a hydrogel layer or mesh arranged between the outer layer and at least one biological element, said hydrogel layer or mesh having a Young's modulus of between 0.01 and 200 kPa, notably between 0.1 and 60 kPa, even more preferentially between 0.1 and 5 kPa.

Preferably, the three-dimensional microcompartment is hollow, and consists of an outer hydrogel layer encapsulating a hollow inner part comprising at least:
one or more biological elements, and
optionally culture medium and/or extracellular matrix elements.

When the inner part of the microcompartment comprises a culture medium, this is adapted to the encapsulated biological elements.

Advantageously, the culture medium provides the nutrients and factors required for the survival, growth and functioning of the biological elements. Thus, the composition of the culture medium can be adjusted based on the specific needs of the encapsulated biological elements.

When the inner part of the microcompartment comprises extracellular matrix elements, these can be selected from proteins such as collagen, fibronectin, laminin, or polysaccharides such as hyaluronic acid and combinations thereof. The presence of extracellular matrix elements can promote the adhesion, the proliferation and the differentiation of encapsulated cells.

The composition of the inner part of the microcompartment can be adapted to create specific microenvironments mimicking different types of tissues or organs. This flexibility means that microcompartments can be used for a variety of applications, such as cell culture, drug testing, regenerative medicine or tissue engineering.

According to another embodiment, the microcompartment is solid, i.e. not hollow, for example a solid hydrogel ball encapsulating at least one biological element.

This solid structure offers greater mechanical resistance with respect to the hollow structure. The solid hydrogel ball also ensures even distribution of biological elements throughout the volume of the microcompartment.

Firstly, the solid hydrogel structure offers greater protection to the encapsulated biological elements. The hydrogel forms a continuous three-dimensional matrix that completely surrounds the biological elements, isolating them from the external environment. This physical barrier can help protect the biological elements from mechanical stress or other potentially damaging factors.

Secondly, the solid ball configuration enables a more uniform distribution of cations throughout the entire volume of the microcompartment. During the rinsing step with a solution containing at least one cation, the latter can diffuse homogeneously throughout the structure of the hydrogel. This uniform diffusion of cations results in a more balanced mechanical reinforcement of the entire microcompartment.

The microcompartment can be formed from different types of hydrogels, each having specific properties suited to different applications.

According to one embodiment, the hydrogel is selected from alginates, carrageenans, pectins, gelatins, collagen, hyaluronic acid, chitosan and combinations thereof.

According to a preferred embodiment, the hydrogel comprises or consists exclusively of alginate.

The alginate may in particular be a sodium alginate, composed of 80% α-L-guluronate and 20% β-D-mannuronate, having a Young's modulus greater than 10 kPa, preferentially greater than 60 kPa, more preferentially greater than 100 kPa. According to one embodiment, the outer hydrogel layer comprises alginate, said alginate having an average molecular weight of 100 to 400 kDa, more preferentially a molecular weight between 150 and 250 kDa.

When the hydrogel of the outer layer of the microcompartment is alginate, the concentration of the alginate solution intended to form said outer layer of the microcompartment is preferentially between 0.5 and 5% by mass, more preferentially the concentration is equal to 2% (plus or minus 0.5%) by mass.

Advantageously, the use of a rinsing solution containing at least one cation, notably divalent cations such as calcium, reinforces the structure of the already crosslinked alginate hydrogel forming the microcompartment.

The cations in the rinsing solution bind to the alginate chains, increasing the crosslinking density and improving the mechanical properties of the microcompartment.

According to one embodiment, at least one biological element encapsulated in the microcompartment is selected from a cell, a cell organelle, a cell fragment, a cell aggregate, an organoid, DNA, RNA, a protein, a microorganism, a vector, a vesicle, an exosome, a microtissue, a spheroid, an embryoid body and combinations thereof.

The choice of biological elements depends on the intended application and the desired properties of the microcompartment.

When the microcompartment comprises at least one cell, this can be any cell type, in particular at least one cell is a eukaryotic cell. More preferentially, at least one cell is a human or plant or animal cell.

In a particular embodiment, the microcompartment comprises pluripotent stem cells. A pluripotent stem cell, or pluripotent cell, refers to a cell that has the ability to form all the tissues present in the entire organism of origin, without however being able to form an entire organism as such. Pluripotent stem cells can be, in particular, induced pluripotent stem (iPS) cells, MUSE (“Multilineage-differentiating Stress Enduring”) cells found in the skin and the bone marrow of adult mammals, or embryonic stem (ES) cells. According to one embodiment, the microcompartment according to the invention does not comprise embryonic stem (ES) cells.

In a particularly suitable variant of the invention, the microcompartment according to the invention comprises human or animal induced pluripotent stem cells.

In another particular embodiment, the microcompartment according to the invention comprises human or animal multipotent cells and/or human or animal progenitor cells derived from these multipotent cells and/or cells undergoing differentiation. The multipotent and/or progenitor cells have preferentially been obtained from pluripotent stem cells, in particular human pluripotent stem cells, or optionally from non-pluripotent human cells whose transcriptional profile has been artificially modified to match that of particular multipotent and/or progenitor cells, typically by forced expression of transcription factors specific to the target cell phenotype. Preferentially, the multipotent and/or progenitor cells have been obtained from pluripotent stem cells after contact with a solution capable of initiating the differentiation of said stem cells.

According to another variant, the microcompartment according to the invention comprises human or animal differentiated cells. The differentiated cells have preferentially been obtained from pluripotent stem cells or progenitor cells, in particular human pluripotent stem cells or human progenitor cells, or optionally from non-pluripotent human cells whose transcriptional profile has been artificially modified to match that of particular differentiated cells, typically by forced expression of transcription factors specific to the target cell phenotype. Preferentially, the differentiated cells have been obtained from pluripotent or multipotent or progenitor stem cells after contact with a solution capable of initiating the differentiation of said stem cells. According to one variant, the cellular content of the microcompartment comprises homogeneous or mixed cellular identities.

The differentiated cells can in particular be in the form of at least one layer of cells or in the form of a three-dimensional tissue, cell aggregate or microtissue, or in the form of several tissues or microtissues in the microcompartment. It can be a compacted or uncompacted tissue or microtissue, with or without lumen.

The microcompartment may therefore comprise several cell types. In particular, the microcompartment may comprise, for example, induced pluripotent stem cells and/or multipotent cells and/or progenitor cells and/or differentiating cells and/or differentiated cells.

Thus, when the microcompartment comprises at least one cell, it is selected from stem cells, progenitor cells, differentiated cells, cancer cells and combinations thereof.

According to one variant, the microcompartment comprises:
an outer hydrogel layer, and
an inner part comprising at least:
   at least one layer of cells, and
   a hydrogel layer or mesh arranged between the outer layer and the layer of cells.

According to this variant, the layer or mesh of the inner hydrogel part, preferentially made of alginate, can comprise other constituents, thus said layer or mesh of the inner hydrogel part preferentially comprises at least one peptide sequence, more preferentially a peptide sequence of interest capable of interacting with the cells notably constituting the layer of cells present in the microcompartment. For example, the peptide sequence may be a peptide or a protein. According to a particularly preferred object, the peptide sequence is a YIGSR motif and/or an RGD motif. The YIGSR motif is a peptide derived from the β1 chain of laminin with a Tyrosine-Isoleucine-Glycine-Serine-Arginine sequence, facilitating cell adhesion to the layer or mesh of the inner hydrogel part. The RGD motif is a peptide with an Arginine-Glycine-Asparagine sequence that also facilitates cell adhesion to the or mesh of the inner hydrogel part.

According to the same variant, the layer or the mesh of the inner hydrogel part may comprise at least a second hydrogel distinct from the first hydrogel of the layer or mesh of the inner part, more preferentially it is selected from fibrin, laminin, fibronectin, entactin, hyaluronic acid and collagen.

In this embodiment, the method according to the invention relates to the treatment of a three-dimensional microcompartment, delimited by the outer hydrogel layer and inside said outer layer, an inner part comprises at least one cell and the hydrogel layer or mesh.

According to one embodiment, the microcompartment encapsulates at least one human biological element.

According to one embodiment, the microcompartment is enclosed. A closed microcompartment offers several advantages for encapsulating biological elements.

The enclosed configuration creates a protective barrier that isolates the encapsulated biological elements from the external environment. This isolation protects the biological elements from potentially harmful external factors, such as contaminants or undesirable chemical or biological agents.

The enclosed nature of the microcompartment also prevents leakages or the escape of encapsulated biological elements. This retention is beneficial for maintaining the desired concentration and location of biological elements such as cells within the microcompartment.

Additionally, an enclosed microcompartment configuration facilitates the handling and the treatment of encapsulated biological elements as a single unit. The closed structure allows the microcompartment to be transferred, washed or subjected to various treatments while keeping the internal contents intact and protected.

According to one embodiment, the microcompartment has the shape of an ovoid, a cylinder, a spheroid, a sphere, a fiber or a teardrop.

Each of these geometric shapes has specific characteristics in terms of mechanical strength, exchange surface and hydrodynamic behavior. The choice of the shape of the microcompartment can be adapted based on the particular requirements of the intended application.

According to one embodiment, the smallest dimension of the microcompartment is between 1 μm and 1.5 mm, preferentially between 100 and 800 μm, even more preferentially between 200 and 700 μm.

According to one embodiment, the method according to the invention also comprises a step of mechanically stressing said rinsed microcompartment, wherein the mechanical stressing is performed by filtration, agitation in liquid medium, centrifugation, suction or a combination thereof.

Advantageously, the rinsing step reinforces the microcompartments, enabling them to better withstand the mechanical stresses applied thereto.

Indeed, the properties of crosslinked microcompartments can be altered by their environment, for example by the constitution of the culture medium in which they are found. The invention reinforces the mechanical properties of microcompartments which increases the production potential on an industrial scale by reducing the limitations associated with their fragility. Thus, the microcompartments can be subjected to steps such as filtration, suction or agitation while losses are limited or eliminated altogether.

According to one embodiment, the mechanical stress step is performed simultaneously with the microcompartment rinsing step.

According to one embodiment, the mechanical stress step applies a centrifugal acceleration of at least 10 g to at least one rinsed microcompartment.

Advantageously, when the mechanical stress step uses the centrifugal effect, by any means, it optimizes the culture conditions in a three-dimensional system.

Indeed, the centrifugal effect promotes the formation of clusters or aggregates of biological elements, preferentially of cells, within microcompartments in order to improve cell survival and growth, as well as the appearance of better cell structuring for the initiation of 3D amplification.

Preferably, when the microcompartments comprise cells and are the result of the mechanical stress step applying a centrifugal acceleration of at least 10 g, they comprise less than 5% single cells by number of the total encapsulated cells, preferentially less than 1%.

Indeed, in the context of the invention, it is preferable for encapsulated cells to be in the form of clusters in order to optimize amplification and improve cell survival.

Advantageously, the centrifugal acceleration of at least 10 g is adapted to promote the appearance of cell clusters without affecting cell viability.

In the context of the invention, the centrifugal acceleration exerted on at least one biological element and/or at least one microcompartment can be calculated by the following formula:

$$G = R * {Ω}^{2} \qquad [ Math 1 ]$$

wherein R is the radius of the trajectory expressed in meters, Ω is the angular velocity expressed in radians per second and G is the centrifugal acceleration expressed in m·s−2

Preferentially, the centrifugal acceleration applied to at least one biological element and/or at least one microcompartment is between 10 and 3000 g, even more preferentially between 50 and 1000, notably between 200 and 400 g.

According to one embodiment, the centrifugal acceleration applied to at least one biological element and/or at least one microcompartment is between 10 and 500 g, preferentially between 10 and 400 g.

According to one embodiment, the centrifugal acceleration is applied by centrifugation, notably using a centrifuge.

According to another embodiment, the centrifugal acceleration is applied using a filtration device.

According to one embodiment, the rinsing step and/or the mechanical stress step can be carried out using at least one device comprising a body (2) having at least a first port (5) and a second port (6) and a filtering membrane (3) extending along a plane P in the body (2), thus defining two compartments and able to prevent the passage of microcompartments from one compartment to the other, the first port (5) being intended for the injection and withdrawal of the cellular microcompartments immersed in a solution A and/or a rinsing solution and the second port (6) being intended for the withdrawal and injection of solution A and/or the rinsing solution, characterized in that the injection into the first port (5) takes place in a direction D that is not substantially orthogonal to the plane P of the filtering membrane (3).

By way of illustration, said device is designated by reference 1 as a whole and shown in FIG. 1 relates to a filtration device comprising a body 2 comprising a filtering membrane 3 which extends into the body 2 along a plane P.

According to one embodiment, the rinsing and mechanical stress steps are carried out simultaneously using at least one device 1.

According to one embodiment, the body 2 of the device 1 comprises an inner casing 4 comprising rounded edges.

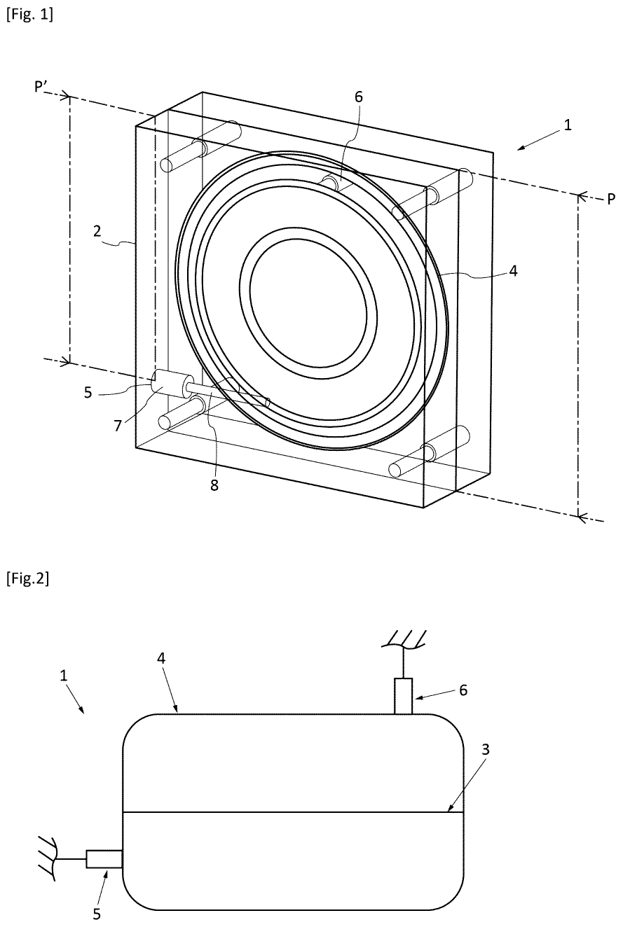

According to one embodiment of the invention notably shown in FIGS. 2 and 5, the inner casing 4 is burger-shaped, that is cylindrical with a rounded upper edge surface and a rounded lower edge surface.

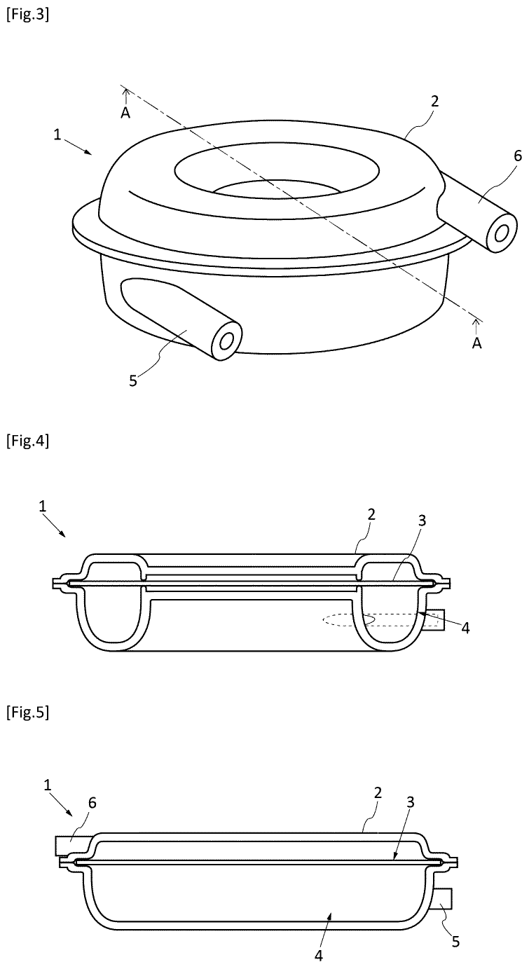

According to the embodiment shown in FIGS. 3 and 4, the inner casing 3 of the body 2 is donut-shaped, that is toroidal.

According to the embodiment shown in FIG. 2, the device 1 is symmetrical with respect to the filtering membrane 3. Other non-symmetrical embodiments shown in FIGS. 3, 4 and 5 are also compatible with the invention. For example, the part intended to receive microcompartments may comprise larger volumetric dimensions than the part on the other side of the filtering membrane 3 or vice versa.

The filtering membrane 3 extends along a plane P through the body 2 of the device 1. The filtering membrane 3 extends up to the edges of the inner casing 4 of the body 2 of the device 1. According to the embodiments shown, the effective cross-section of the filtering membrane 3 is circular. According to other embodiments not shown, the effective cross-section of the filtering membrane 3 is elliptical.

In order to retain the microcompartments, the filtering membrane 3 comprises a fibrous membrane capable of preventing microcompartments from passing through.

The filtering membrane 3 is therefore adapted to the type of microcompartments used.

According to one embodiment of the invention, the filtering membrane 3 comprises a microtissue covered with a fibrous membrane. The fibrous membrane contains material such as mesh, nylon or PET. Other microcompartment-filtering materials can be used within the scope of the invention.

According to one embodiment of the invention, for microcompartments having a larger dimension of between 120 and 700 micrometers, the filtering membrane comprises porosities of less than 100 micrometers.

According to one embodiment of the invention, for microcompartments having a largest dimension of less than 600 micrometers, the filtering membrane comprises porosities of less than 100 micrometers.

According to one embodiment of the invention, the filtering membrane 3 is suitable for filtering at least one type of microcompartment and is also suitable for allowing through at least one other type of microcompartment. The mesh can thus be adapted to the dimensions of the microcompartments to be filtered.

The body 2 of the device 1 comprises a first port 5 for tangential injection of microcompartments, immersed in a soaking solution.

For this purpose, the first port 5 has a cylindrical shape which extends substantially in a direction D belonging to a plane P′ parallel to the plane P of the filtering membrane 3 as shown in FIG. 1.

The second port 6 is located in the second part of the body 2, on the other side of the filtering membrane 3. According to the embodiment shown in FIGS. 1 and 2, the second port 6 does not allow tangential injection into the body 2 of the device 1. According to the embodiment notably shown in FIGS. 3, 4 and 5, the second port 6 is also suitable for tangential injection into the body 2 of the device 1.

According to the embodiment shown in FIG. 1, the first port 5 comprises an inlet 7 and an injection channel 8.

The injection channel 8 enables tangential injection into the inner casing 4 of the body 2 of the device 1.

According to other embodiments not shown, the body 2 of the device 1 comprises more than two inlet and outlet ports, thus enabling different types of solution of microcompartments to be injected on each side of the filtering membrane 3.

Other forms of architecture can be implemented depending on the use of the invention.

In the context of the invention, solution A can be any type of solution likely to comprise microcompartments.

According to one embodiment, solution A is a culture medium suitable for maintaining the microcompartments in suspension or a crosslinking solution.

According to one embodiment, the rinsing and mechanical stress steps are carried out continuously using at least 2 devices 1 according to any one of the preceding embodiments. Preferentially, when the rinsing and mechanical stress steps are carried out continuously using at least 2 devices 1 according to any one of the preceding embodiments, said devices are arranged in series.

### Method for Preparing a Microcompartment:

According to another object, the invention relates to a method for preparing a three-dimensional microcompartment comprising the implementation of the following steps:
a) Encapsulation of at least one biological element in a crosslinked hydrogel to form a three-dimensional microcompartment; and
b) Treatment of the microcompartment obtained in step a) according to the treatment method of any one of the preceding embodiments.

Advantageously, the preparation method according to the invention makes it possible to form a microcompartment having improved resistance to mechanical stress, notably during filtration, centrifugation, suction or combinations thereof.

Preferentially, at least one three-dimensional microcompartment obtained from step a) is hollow in the sense that it has an outer layer and a hollow inner part comprising at least one biological element. Preferably, at least one microcompartment obtained from step a) has an outer hydrogel layer and an inner part comprising at least one cell.

According to one embodiment, the microcompartment obtained from step a) has an outer hydrogel layer and an inner part comprising an internal matrix and at least one biological element, preferentially a cell. Preferentially, the internal matrix is an extracellular matrix or extracellular matrix substitute.

The microcompartment from step a) comprises at least one biological element, preferentially at least one cell, encapsulated in hydrogel, said hydrogel being biocompatible, that is non-toxic to the biological elements, preferentially to the cells. The hydrogel must allow the diffusion of oxygen and nutrients to feed the biological elements when necessary, notably for the cells contained in the microcompartment and to enable their survival. According to a particularly preferred embodiment, the outer hydrogel layer of the microcompartment from step a) comprises at least alginate. According to a particular embodiment, the outer hydrogel layer of the microcompartment from step a) can be made exclusively of alginate.

The alginate may in particular be a sodium alginate, composed of 80% α-L-guluronate and 20% β-D-mannuronate, having a Young's modulus greater than 10 kPa, preferentially greater than 60 kPa, more preferentially greater than 100 kPa.

According to one embodiment, the outer hydrogel layer of the microcompartment from step a) comprises alginate, said alginate advantageously having an average molecular weight of 100 to 400 kDa, more preferentially the outer layer has a molecular weight of between 150 and 250 kDa. When the hydrogel of the outer layer of the microcompartment from step a) is alginate, the concentration of the alginate solution intended to form said outer layer of the microcompartment is preferentially between 0.5 and 5% by mass, more preferentially the concentration is equal to 2% (plus or minus 0.5%) by mass.

When the concentration of the alginate solution intended to form the outer layer of the microcompartment in step a) is equal to 2%, the viscosity of the alginate is preferentially between 500 and 1600 mPa/s, more preferentially between 1000 and 1400 mPa/s.

Advantageously, the outer hydrogel layer of the microcompartment from step a) is devoid of biological elements, preferentially devoid of cells.

The outer hydrogel layer of the microcompartment from step a) thus protects the biological elements, preferentially the cells, from the external environment. When the microcompartment from step a) comprises cells, the outer hydrogel layer also makes it possible to limit cell multiplication through this physical barrier.

According to one embodiment, the microcompartment from step a) comprises at least one biological element selected from: a cell, a cell organelle, a cell fragment, a cell aggregate, an organoid, DNA, RNA, a protein, a microorganism, a vector, a vesicle, an exosome, a microtissue, a spheroid, an embryoid body and combinations thereof.

When the microcompartment from step a) comprises cells, the cells can be any cell type, in particular the cells are eukaryotic cells. More preferentially, the cells are human or plant or animal cells.

In a particular embodiment, the microcompartment from step a) comprises pluripotent stem cells. A pluripotent stem cell, or pluripotent cell, refers to a cell that has the ability to form all the tissues present in the entire organism of origin, without however being able to form an entire organism as such. Pluripotent stem cells can be, in particular, induced pluripotent stem (iPS) cells, MUSE (“Multilineage-differentiating Stress Enduring”) cells found in the skin and the bone marrow of adult mammals, or embryonic stem (ES) cells. According to one embodiment, the microcompartment according to the invention does not comprise embryonic stem (ES) cells.

According to a particularly suitable variant of the invention, the microcompartment from step a) comprises human or animal induced pluripotent stem cells.

In another particular embodiment, the microcompartment from step a) comprises human or animal multipotent cells and/or human or animal progenitor cells derived from these multipotent cells and/or cells undergoing differentiation. The multipotent and/or progenitor cells have preferentially been obtained from pluripotent stem cells, in particular human pluripotent stem cells, or optionally from non-pluripotent human cells whose transcriptional profile has been artificially modified to match that of particular multipotent and/or progenitor cells, typically by forced expression of transcription factors specific to the target cell phenotype. Preferentially, the multipotent and/or progenitor cells have been obtained from pluripotent stem cells after contact with a solution capable of initiating the differentiation of said stem cells.

According to another variant, the microcompartment from step a) comprises human or animal differentiated cells. The differentiated cells have preferentially been obtained from pluripotent stem cells or progenitor cells, in particular human pluripotent stem cells or human progenitor cells, or optionally from non-pluripotent human cells whose transcriptional profile has been artificially modified to match that of particular differentiated cells, typically by forced expression of transcription factors specific to the target cell phenotype. Preferentially, the differentiated cells have been obtained from pluripotent or multipotent or progenitor stem cells after contact with a solution capable of initiating the differentiation of said stem cells. According to one variant, the cellular content of the microcompartment comprises homogeneous or mixed cellular identities.

The differentiated cells can in particular be in the form of at least one layer of cells or in the form of a three-dimensional tissue, cell aggregate or microtissue, or in the form of several tissues or microtissues in the microcompartment. It can be a compacted or uncompacted tissue or microtissue, with or without lumen.

The microcompartment from step a) can therefore comprise several cell types. In particular, the microcompartment from step a) may comprise, for example, induced pluripotent stem cells and/or multipotent cells and/or progenitor cells and/or differentiating cells and/or differentiated cells.

According to one variant, the microcompartment from step a) comprises:
an outer hydrogel layer, and
an inner part comprising at least:
   at least one layer of cells, and
   a hydrogel layer or mesh arranged between the outer layer and the layer of cells.

According to this variant, the layer or mesh of the inner hydrogel part, preferentially made of alginate, can comprise other constituents, thus said layer or mesh of the inner hydrogel part preferentially comprises at least one peptide sequence, more preferentially a peptide sequence of interest capable of interacting with the cells notably constituting the layer of cells present in the microcompartment from step a). For example, the peptide sequence may be a peptide or a protein. According to a particularly preferred object, the peptide sequence is a YIGSR motif and/or an RGD motif. The YIGSR motif is a peptide derived from the β1 chain of laminin with a Tyrosine-Isoleucine-Glycine-Serine-Arginine sequence, facilitating cell adhesion to the layer or mesh of the inner hydrogel part. The RGD motif is a peptide with an Arginine-Glycine-Asparagine sequence that also facilitates cell adhesion to the or mesh of the inner hydrogel part.

According to the same variant, the layer or the mesh of the inner hydrogel part may comprise at least a second hydrogel distinct from the first hydrogel of the layer or mesh of the inner part, more preferentially it is selected from fibrin, laminin, fibronectin, entactin, hyaluronic acid and collagen.

The microcompartment obtained from step a) is then a three-dimensional microcompartment, delimited by the outer hydrogel layer and, inside said outer layer, an inner part comprises at least one cell and the hydrogel layer or mesh.

The microcompartment obtained from step a) is preferentially in various spherical or substantially spherical shapes. Advantageously, the three-dimensional microcompartment is hollow, more preferentially, the hollow microcompartment is in the shape of an ovoid, a cylinder, a spheroid, a sphere or a teardrop or in a substantially ovoid, substantially cylindrical, substantially spheroid, substantially spherical or substantially teardrop shape.

On the one hand, the outer hydrogel layer protects the cells from the external environment, limiting uncontrolled cell proliferation and, in the event of differentiation, cell differentiation. On the other hand, the optional presence of a layer or mesh in the inner hydrogel part and/or the extracellular matrix and/or extracellular matrix substitute provides a suitable environment for cell growth and multiplication.

According to one embodiment, the microcompartment from step a) has a larger dimension of between 1 μm and 1.5 mm, preferentially between 120 μm and 800 μm, preferentially between 200 μm and 600 μm, even more preferentially 200 μm and 500 μm, notably between 200 μm and 250 μm.

According to one embodiment, the microcompartment from step a) has a larger dimension of between 400 and 500 μm.

According to one embodiment, the microcompartment from step a) has a smaller dimension of between 1 μm and 1.5 mm, preferentially between 100 μm and 800 μm.

In the context of the invention, the largest dimension of the microcompartment is always greater than the smallest dimension of the microcompartment.

Thus, according to one embodiment, the microcompartment from step a) has:
a larger dimension of between 1 μm and 1.5 mm, preferentially between 120 μm and 800 μm, preferentially between 200 μm and 600 μm, even more preferentially 200 μm and 500 μm, notably between 200 μm and 250 μm; and/or
a smaller dimension of between 1 μm and 1.5 mm, preferentially between 100 μm and 800 μm.

According to one embodiment, encapsulation step a) comprises a hydrogel crosslinking step performed using a crosslinking solution comprising at least one crosslinking agent.

According to one embodiment, step a) may comprise the following steps:
1) forming a hydrogel microcompartment, preferentially hollow, encapsulating biological elements, using an encapsulation device, preferentially comprising a micro-fluidic and/or milli-fluidic device and solutions, said solutions being preferentially the following:
   a hydrogel solution;
   optionally an intermediate solution, preferentially isotonic; and
   a solution comprising biological elements, culture medium and, optionally, the extracellular matrix and/or extracellular matrix substitute;
2) crosslinking the hydrogel, preferentially using a crosslinking solution, such as a crosslinking bath.

Preferentially, step a) of encapsulating at least one biological element in a crosslinked hydrogel comprises the implementation of the following steps:
1) bringing at least one biological element, preferentially at least one cell, into contact with a hydrogel solution intended to form an outer layer to form at least one drop, and
2) Crosslinking the hydrogel using a crosslinking solution capable of crosslinking said hydrogel solution to form the outer layer of each microcompartment.

According to one embodiment, encapsulation step a) comprises the following steps:
1) co-injecting two or three solutions to form a hollow hydrogel microcompartment encapsulating biological elements, said solutions being selected from:
   a hydrogel solution;
   optionally an intermediate solution, preferentially isotonic;
   a solution comprising biological elements, culture medium and optionally extracellular matrix and/or extracellular matrix substitute, concentrically via a micro-fluidic injector which forms a jet at the injector outlet consisting of the mixture of solutions, said jet breaking up into drops;
2) Crosslinking the hydrogel.

Preferentially, step a) of encapsulating at least one biological element in a crosslinked hydrogel comprises the implementation of the following steps:
1) bringing at least one biological element, preferentially at least one cell, into contact with a hydrogel solution intended to form an outer layer to form at least one drop, and
2) collecting the resulting drop in a crosslinking solution capable of crosslinking said hydrogel solution to form the outer layer of each microcompartment.

Once the outer hydrogel layer has been crosslinked by the crosslinking solution, the microcompartment is formed.

According to one embodiment, encapsulation step a) is carried out by co-injecting two or three solutions:
a hydrogel solution;
optionally an intermediate solution, preferentially isotonic;
a solution comprising biological elements, preferentially cells, culture medium and optionally extracellular matrix and/or an extracellular matrix substitute, concentrically via a micro-fluidic injector which forms a jet at the injector outlet consisting of the mixture of solutions, said jet breaking up into drops.

In this embodiment, in step 2) the drops formed in step 1) are collected in a crosslinking solution which crosslinks the hydrogel solution to form the outer layer of each microcompartment, the inner part of each drop consisting of the solution comprising the biological elements, preferentially the cells, of the culture medium and the extracellular matrix.

Thus, according to a preferred embodiment, the method according to the invention comprises an encapsulation step a) comprising the implementation of the following steps:
1) Encapsulating at least one biological element, preferentially a cell, in hydrogel so as to form a suspension comprising at least one microcompartment in a crosslinking solution, said encapsulation being carried out by co-injecting two or three solutions:
   a hydrogel solution;
   optionally an intermediate solution, preferentially isotonic;
   a solution comprising biological elements, preferentially cells, culture medium and optionally extracellular matrix and/or an extracellular matrix substitute, concentrically via a micro-fluidic injector which forms a jet at the injector outlet consisting of the mixture of solutions, said jet breaking up into drops,
2) collecting said drops in a crosslinking solution which crosslinks the hydrogel solution to form the outer layer of each microcompartment, the inner part of each drop consisting of the solution comprising the biological elements, preferentially cells, culture medium and optionally extracellular matrix.

In this embodiment, the intermediate, preferentially isotonic, solution of encapsulation step 1) is preferentially a sorbitol solution.

In the same embodiment, the final opening diameter of the micro-fluidic injector in encapsulation step 1) is preferentially between 50 and 800 μm, preferentially between 80 and 240 μm, and the flow rate of each of the solutions is between 0.1 and 2000 mL/h, preferentially between 10 and 2000 mL/h, more preferentially between 11 and 100 mL/h.

According to one embodiment, the crosslinking solution comprises calcium.

Preferably, the crosslinking solution comprises calcium at a concentration of between 0.5 and 300 mM, preferentially between 25 and 150 mM, even more preferentially between 50 and 120 mM.

According to one embodiment, the crosslinking solution comprises at least one cation, preferentially divalent, having a concentration of at least 25 mM, preferably at least 50 mM.

According to one embodiment, at least one crosslinking agent from step a) is distinct from at least one cation from step b).

According to another embodiment, at least one crosslinking agent from step a) is identical to at least one cation from step b).

According to a preferred embodiment, the crosslinking agent from step a) and the cation from step b) is calcium.

According to one embodiment, step a) can be implemented using an encapsulation system comprising notably preferentially a micro-fluidic or milli-fluidic device.

In one embodiment, step a) is carried out using an encapsulation system comprising at least one member capable of electrically charging at least one of the solutions with an electrical potential and the encapsulation device comprises a body arranged to form a concentric flow from the solutions provided by the dispenser(s), an external flow of which is a hydrogel solution and an internal flow is a solution comprising biological elements, and a nozzle connected to the body to receive said concentric flow and forming the outlet of the encapsulation device, the encapsulation device being arranged to form, at the outlet of the nozzle, a concentric jet from the concentric flow so that this jet breaks up into microcompartments.

In this embodiment, the encapsulation device may be a micro-fluidic or milli-fluidic type device capable of generating a concentric jet comprising at the center a solution comprising biological elements, where appropriate surrounded by the intermediate solution, itself where appropriate surrounded by the hydrogel solution.

The increase in hydrodynamic instabilities in the jet forces the jet to break up into drops, this effect being known as the Plateau-Rayleigh instability.

These drops, once the hydrogel solution has crosslinked, form the microcompartments. Electrically charging at least one of the solutions passing through the encapsulation device makes it possible to improve the break-up of the jet into drops. This technique is notably referred to as “electro-jetting”. Note that the relative sizes of the outer layer and of the inner part of the microcompartments can be adjusted by modifying the flow rate ratios of the two solutions at the dispensers.

In the case of electro-jetting, it may be optionally provided to add a member for generating an electric field, such as a metal ring arranged downstream of the outlet of the encapsulation device so that the jet or the microcompartments pass through this ring. Where appropriate, the member for generating an electric field may be connected to an electric potential, for example, to the ground. This electric field notably makes it possible to promote the dispersion of microcompartments.

In another embodiment, step a) is carried out using an encapsulation system comprising at least one member capable of electrically charging at least one of the solutions with an electrical potential and the encapsulation device comprises a body arranged to form a concentric flow from the solutions provided by the dispenser(s), an external flow of which is the hydrogel solution and an internal flow is the solution comprising biological elements, and a nozzle connected to the body to receive said concentric flow and forming the outlet of the encapsulation device, the encapsulation device being arranged to form, directly at the outlet of the nozzle, drops from the concentric flow, which form, once the hydrogel solution has crosslinked, the microcompartments. The encapsulation device is thus of the “electro-dripping” type, and thus forms the drops one after the other directly from the nozzle, without jet.

In yet another embodiment, step a) is performed using an encapsulation device comprising a body arranged to form a concentric flow from the solutions provided by the dispenser(s), an external flow of which is the hydrogel solution and an internal flow is the solution comprising biological elements, and a nozzle connected to the body to receive said concentric flow and forming the outlet of the encapsulation device, and the encapsulation system also comprises an acoustic wave generator coupled to the nozzle and/or to the body so that the encapsulation device is arranged to form, directly at the outlet of the nozzle, drops from the concentric flow. In this embodiment, the encapsulation device is thus of the “acousto-dripping” type, and forms drops one after the other directly from the nozzle under the effect of the acoustic waves emitted by the generator, the dimensions of the drops being determined by the choice of frequency and amplitude of the acoustic waves.

In yet another embodiment, step a) is performed using an encapsulation device comprising a body arranged to form a concentric flow from the solutions provided by the dispenser(s), an external flow of which is the hydrogel solution and an internal flow is the solution comprising biological elements, and a nozzle connected to the body to receive said concentric flow and forming the outlet of the encapsulation device, and the encapsulation system also comprises a vibrating element coupled to the nozzle and/or to the body so that the encapsulation device is arranged to form, at the outlet of the nozzle, a concentric jet from the concentric flow, said jet breaking up into drops. In this embodiment, the encapsulation device is thus of the “vibrating-jetting” type and forms drops by breaking up the concentric jet by virtue of a Plateau-Rayleigh instability induced by the vibrations generated by the vibrating element, the dimensions of the drops being determined by the choice of frequency and amplitude of these vibrations. Said vibrating element could, for example, be a piezoelectric actuator.

In yet another embodiment, step a) is performed using an encapsulation device comprising a body arranged to form a concentric flow from the solutions provided by the dispenser(s), of which an external flow is the hydrogel solution and an internal flow is the solution comprising biological elements, and a nozzle connected to the body to receive said concentric flow and forming the outlet of the encapsulation device and the encapsulation system also comprises a cutting element arranged downstream of the nozzle outlet, so that the encapsulation device is arranged to form, at the nozzle outlet, a concentric jet from the concentric flow, said jet being broken up by the cutting element. In this embodiment, the encapsulation device is thus of the “Jet cutting” type and forms the drops by cutting the concentric jet using the cutting element. The cutting element may for example be a rotating blade with the size of the drops being determined by the speed of rotation and the size of the rotating blade.

In another embodiment, step a) is performed using an encapsulation device comprising a body arranged to form a concentric flow from the solutions provided by the dispenser(s), of which an external flow is the hydrogel solution and an internal flow is the solution comprising biological elements, and a nozzle connected to the body to receive said concentric flow and forming the outlet of the encapsulation device, and the encapsulation system comprises an electromechanical element coupled to the nozzle, the encapsulation device being arranged to form, directly at the outlet of each nozzle, drops from the concentric flow under the effect of a vibration applied by the electromechanical element.

In this embodiment, the encapsulation device is thus of the “inkjet printing” type, and forms drops one after the other directly from each nozzle. Said electromechanical element could, for example, be a piezoelectric actuator, the dimensions of the drops being determined by the choice of frequency and amplitude of the vibrations applied by this element.

Regardless of the considered embodiment, step a) can be carried out by an encapsulation system comprising a collection tank containing a crosslinking solution, preferentially comprising calcium, and arranged to collect the microcompartments formed by the encapsulation device. Where appropriate, the collection tank can be arranged downstream of the encapsulation device to collect the microcompartments formed by the encapsulation device, the hardening solution being arranged to cause hardening of the outer layer of each microcompartment upon immersion thereof in this solution.

For example, the outlet of the encapsulation device could be positioned above the collection tank, so that the microcompartments fall by gravity into this collection tank. Advantageously, the collection tank and the encapsulation device are arranged at a distance from each other such that the microcompartments formed by the encapsulation device pass through a gaseous volume, notably air, defined by an enclosed and sterile chamber before being collected by the collection tank.

In yet another embodiment, step a) is performed using an encapsulation device comprising a body arranged to form a concentric flow from the solutions provided by the dispenser(s), an external flow of which is the hydrogel solution and an internal flow is the solution comprising biological elements, and a nozzle connected to the body to receive said concentric flow and forming the outlet of the encapsulation device, the encapsulation device and the collection tank being arranged so that the nozzle of the encapsulation device is in contact with a collection fluid, such as oil, contained in a collection tank and/or so that the nozzle of the encapsulation device is immersed in a collection fluid contained in a collection tank, the encapsulation device being arranged to form, directly in the collection fluid, drops or a jet, subsequently breaking up into drops, from the concentric flow. The solution capable of gelling can then be crosslinked, for example via a hardening solution, to form the microcompartments.

Regardless of the considered embodiment, it may be provided that the body and/or the nozzle are made of glass. Otherwise, the body and/or nozzle can be made of polymer or metal. It may be provided that the body and the nozzle form a single part or that the body and the nozzle are made separately then assembled to form the encapsulation device.

Advantageously, the body comprises a first inlet connected to the first dispenser to receive the solution comprising biological elements and at least one second inlet connected to the second dispenser to receive the hydrogel solution, as well as a single outlet connected to the nozzle, the body comprising a main channel comprising a substantially rectilinear portion defining a central axis of the encapsulation device, the main channel connecting the first inlet to the single outlet and at least one secondary channel connecting the second inlet to the single outlet, said secondary channel subdividing into portions extending around the first channel, said subdivisions of the second channel merging at the single outlet into a single circular portion, concentric with the first channel, said single circular portion and the first channel merging to form the single outlet of the body.

In another embodiment of the invention, step a) is carried out using an encapsulation device comprising a first body fitted with a nozzle, the first body being arranged to form at the outlet of the nozzle a first jet of the solution comprising biological elements provided by the first dispenser, this first jet breaking up into drops, and a second body fitted with a nozzle, the second body being arranged to form, at the outlet of the nozzle, a second jet of the hydrogel solution provided by the second dispenser, the first and second bodies being arranged so that the drops from the first jet interact with the second jet to form the microcompartments.

In this embodiment, the first jet is broken up into drops by the Plateau-Rayleigh instability. These drops meet the second continuous jet, which then encapsulates these drops, by the Marangoni effect, to form the microcompartments. The outer layer of the microcompartments, formed by the hydrogel solution, can then be crosslinked in a gaseous environment, such as in air, for example using ultraviolet radiation.

Provision may also be made for any combination of the embodiments previously described, or even other embodiments of the encapsulation device for generating microcompartments without departing from the scope of the present invention, and notably encapsulation devices enabling drops to be formed one after the other at the outlet of the encapsulation device and equipped with a means of controlling the ejection of the drops and controlling the dimensions of the drops as they are ejected, encapsulation devices enabling a concentric jet to be formed at the outlet of the encapsulation device and equipped with a means of separating the jet, after it has left the encapsulation device, into drops, or else encapsulation devices enabling drops or a jet from a first device to be coated with drops or a jet from another device.

According to one embodiment, the preparation method according to the invention also comprises a step of mechanically stressing said rinsed microcompartment, wherein the mechanical stressing is performed by filtration, agitation in liquid medium, centrifugation, suction or a combination thereof.

According to one embodiment, the preparation method according to the invention also comprises a step of culturing the biological elements, preferentially cells, encapsulated in the microcompartment. This culture step is preferentially carried out in a bioreactor.

According to one embodiment, the preparation method also comprises a cryopreservation step of the microcompartments obtained in step b). Cryopreservation enables the long-term storage of microcompartments while preserving the viability of encapsulated cells.

According to one embodiment, the cryopreservation step of the microcompartments obtained in step b) comprises the implementation of the following steps:
Microcompartments obtained in step b) are suspended in a cryopreservative;
Optionally, homogeneous distribution of the microcompartments in the cryopreservative solution;
Gradual lowering of the temperature of the cryopreservative solution comprising the microcompartments to 4° C., to be adapted based on the biological elements contained in the microcompartments
Rapid lowering of the temperature of the cryopreservative solution comprising the microcompartments from −4° C. to −180° C., to be adapted based on the biological elements contained in the microcompartments. This can be, for example, at a rate of 1° C. per minute down to −80° C. in a passive or controlled manner.

### Microcompartment According to the Invention

The invention also relates to a three-dimensional microcompartment, obtained by any one of the embodiments of the preparation method according to the invention or any one of the embodiments of the treatment method according to the invention.

The microcompartment according to the invention thus features unique characteristics combining mechanical strength, biocompatibility and versatility of application, making it particularly suitable for a variety of uses in the biomedical and biotechnological field.

According to one embodiment, the microcompartment according to the invention has a Young's modulus of between 3 kPa and 1.2 MPa, preferentially 3 and 800 kPa, notably between 3 and 600 kPa, between 3 and 500 kPa, preferentially between 3 and 350 kPa.

According to one embodiment, the microcompartment according to the invention has a rupture stress of between 10 and 500 kPa, notably between 200 and 500 kPa.

In the context of the invention, the rupture stress of the microcompartment can be measured using a compression test, a torsion test or an extension test.

According to one embodiment, the microcompartment according to the invention has:
a Young's modulus between 3 kPa and 1.2 MPa, preferentially between 3 and 800 kPa, notably between 3 and 600 kPa, between 3 and 500 kPa, preferentially between 3 and 350 kPa; and/or
a rupture stress of between 10 and 500 kPa, notably between 200 and 500 kPa.

According to one embodiment, the microcompartment according to the invention is hollow and comprises an outer hydrogel layer having a Young's modulus of between 3 kPa and 1.2 MPa, preferentially between 3 and 800 kPa, notably between 3 and 600 kPa, between 3 and 500 kPa, preferentially between 3 and 400 kPa, more preferentially between 3 and 300 kPa and defining an inner part, said inner part comprising at least one biological element.

According to one embodiment, the microcompartment according to the invention comprises:
an outer hydrogel layer, and
an inner part comprising at least:
   at least one biological element, and
   a hydrogel layer or mesh arranged between the outer layer and at least one biological element, said hydrogel layer or mesh having a Young's modulus of between 0.01 and 200 kPa, notably between 0.1 and 60 kPa, even more preferentially between 0.1 and 5 kPa.

According to one embodiment, the microcompartment according to the invention comprises:
an outer hydrogel layer, said hydrogel having a Young's modulus of between 3 kPa and 1.2 MPa, preferentially between 3 and 800 kPa, notably between 3 and 600 kPa. 3 and 500 kPa, preferentially between 3 and 400 kPa, more preferentially between 3 and 300 kPa, and
an inner part comprising at least:
   at least one biological element, and
   a hydrogel layer or mesh arranged between the outer layer and at least one biological element, said hydrogel layer or mesh having a Young's modulus of between 0.01 and 200 kPa, notably between 0.1 and 60 kPa, even more preferentially between 0.1 and 5 kPa.

According to one embodiment, the microcompartment according to the invention is obtained by the preparation method according to the invention comprising the implementation of the following steps:
a) Encapsulating at least one biological element in a crosslinked hydrogel to form a three-dimensional microcompartment comprising the following steps:
1) Encapsulating at least one biological element, preferentially a cell, in hydrogel so as to form a suspension comprising at least one microcompartment in a crosslinking solution, said encapsulation being carried out by co-injecting two or three solutions:
   a hydrogel solution;
   optionally an intermediate solution, preferentially isotonic;
   a solution comprising biological elements, preferentially cells, culture medium and optionally extracellular matrix and/or an extracellular matrix substitute, concentrically via a micro-fluidic injector which forms a jet at the injector outlet consisting of the mixture of solutions, said jet breaking up into drops,
2) Crosslinking the hydrogel; and
b) Treatment of the microcompartment obtained in step a) by a method comprising a step of rinsing the microcompartment in a rinsing solution comprising at least one cation, preferentially comprising calcium, and more preferentially comprising calcium at a concentration of between 0.5 and 10 mM.

### Use of the Microcompartment According to the Invention

The invention also relates to the use of a microcompartment according to the invention for cell culture, drug testing, regenerative medicine or tissue engineering.

### Kit

The invention also relates to a kit for implementing the preparation or treatment method according to the invention comprising at least one rinsing solution containing at least one cation.

Advantageously, the kit according to the invention provides the user with a suitable solution for enhancing the mechanical properties of three-dimensional microcompartments comprising crosslinked hydrogel.

According to one embodiment, the concentration of at least one cation in the rinsing solution is between 0.5 and 50 mM.

Preferably, at least one cation in the rinsing solution can be selected from calcium, iron, barium, strontium, copper, lead, aluminum, magnesium, manganese, zinc, cobalt, nickel and combinations thereof.

According to a particular embodiment, the kit according to the invention comprises a rinsing solution comprising at least 2, 3, 4, 5 distinct cations.

Preferentially, at least one cation in the rinsing solution is a divalent or trivalent cation.

According to a particular embodiment, the rinsing solution in the kit comprises at least one divalent cation and at least one trivalent cation.

The rinsing solution in the kit can vary in terms of composition and cation concentration.

According to one embodiment, the rinsing solution in the kit is a solution in which at least one cation has a concentration of between 0.5 and 50 mM, notably between 0.5 and 25 mM.

Advantageously, within the range of values between 0.5 and 50 mM, different concentrations can be used according to the specific needs based on the cations used.

The concentration of cations in the rinsing solution in the kit can be adjusted within the concentration range described based on the specific needs of the microcompartment and the biological elements contained therein. A higher concentration of cations may lead to greater reinforcement of the mechanical properties, while a lower concentration may be preferable to maintain the viability of certain sensitive biological elements.

Preferably, the rinsing solution in the kit comprises at least calcium. According to a particular embodiment, the rinsing solution comprises only one type of cation: calcium.

Preferentially, the rinsing solution in the kit comprises at least one divalent calcium (Ca²⁺).

Particularly suitably, the rinsing solution comprises calcium at a concentration of between 0.5 and 10 mM, preferentially between 2.5 and 5 mM.

In particular, the rinsing solution can comprise calcium at a concentration of between 1 and 8 mM, preferentially between 1 and 6 mM, more preferentially between 2 and 6 mM, even more preferentially between 2.5 and 5 mM.

According to one embodiment, the rinsing solution comprises calcium in a concentration of between 0.5 and 2 mM, notably between 0.5 and 1 mM.

According to another embodiment, the rinsing solution comprises at least iron. According to a particular embodiment, the rinsing solution comprises only one type of cation: iron.

Particularly suitably, the rinsing solution comprises iron at a concentration of between 0.5 and 10 mM, preferentially between 0.5 and 5 mM.

According to one embodiment, the rinsing solution comprises a ferric cation (Fe²⁺) and/or a ferrous cation (Fe³⁺).

According to another embodiment, the rinsing solution comprises calcium and at least one cation selected from iron (preferentially Fe²⁺ and/or Fe³⁺), barium, strontium, copper, lead, aluminum, magnesium, manganese, zinc, cobalt, nickel ions and combinations thereof.

According to one embodiment, the rinsing solution comprises between 0.5 and 10 mM of calcium and at least one cation selected from iron (preferentially Fe²⁺ and/or Fe³⁺), barium, strontium, copper, lead, aluminum, magnesium, manganese, zinc, cobalt, nickel ions and combinations thereof at a concentration of between 0.5 and 50 mM, notably between 0.5 and 25 mM, preferentially between 0.5 and 10 mM, more preferentially between 0.5 and 5 mM.

According to one embodiment, the rinsing solution comprises calcium ions (preferentially Ca²⁺) and iron ions (preferentially Fe²⁺ and/or Fe³⁺).

Preferentially, in this embodiment, the rinsing solution comprises between 0.5 and 10 mM of calcium (preferentially Ca²⁺) and between 0.5 and 9 mM of iron (preferentially Fe²⁺ and/or Fe³⁺).

According to one embodiment, the kit according to the invention comprises a rinsing solution comprising calcium at a concentration of between 0.5 and 10 mM.

According to one embodiment, the kit may comprise least two rinsing solutions with different cation concentrations, enabling the method to be adjusted according to the specific needs.

Thus, according to this embodiment, the kit according to the invention comprises a rinsing solution comprising calcium having a concentration of 0.5 and 10 mM and another rinsing solution comprising at least one cation selected from iron, barium, strontium, copper, lead, aluminum, magnesium, manganese, zinc, cobalt, nickel and combinations thereof having a concentration of between 0.5 and 50 mM.

According to one embodiment, the kit according to the invention comprises a rinsing solution comprising calcium having a concentration of 0.5 to 10 mM and another rinsing solution comprising iron having a concentration between 0.5 and 5 mM.

### Examples

### Example 1: Demonstrating the Effect of the Treatment Method According To the Invention (Calcium) on the Mechanical Strength of Microcompartments, in the Context of Filtration Using a Cell Sieve

A 2.6 mL volume of microcompartments was deposited in a reversible cell sieve with a porosity of 100 μm.

A rinsing solution was used, comprising a culture medium, DMEM/F-12 initially comprising about 0.9 mM of calcium, supplemented or not with calcium at a concentration of 2 mM CaCl₂). Thus, the calcium concentration of the rinsing solution comprising calcium-supplemented DMEM/F-12 culture medium is 2.9 mM.

The microcompartments were rinsed at a flow rate of 50 mL/min for 1 minute.

### Results

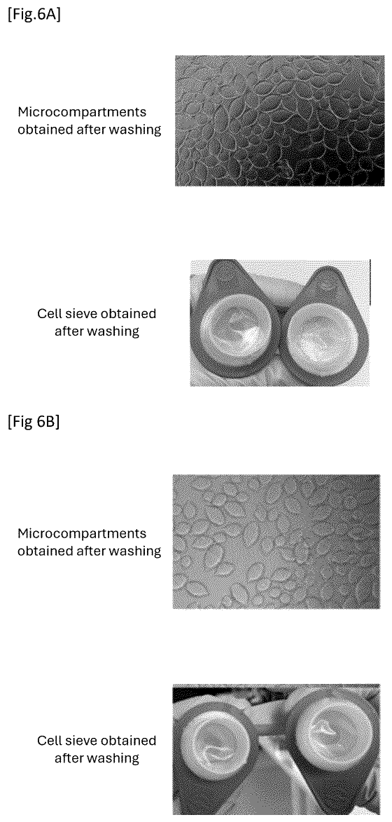

The results of this example are shown in FIGS. 6A and 6B:

FIG. 6A: In the absence of calcium supplementation, the cellular microcompartments show increased fragility, as well as a greater tendency to agglomerate on the filter.

FIG. 68: Conversely, the microcompartments washed with the calcium-supplemented solution show improved mechanical strength and reduced agglomeration on the filter.

This example highlights the beneficial impact of the treatment method according to the invention via a rinsing step using a rinsing solution comprising calcium on the strength and the stability of cellular microcompartments during handling.

### Example 2: Demonstrating the Effect of the Treatment Method According to the Invention (Calcium) on the Mechanical Strength of the Microcompartments, in the Context of Rinsing and Filtering Using the Device 1

A 4 mL volume of microcompartments was introduced into the device 1. The microcompartments were then rinsed with DMEM/F-12 supplemented with different concentrations of calcium, ranging from 2 mM to 4 mM, for 15 seconds at a flow rate of 250 mL/min.

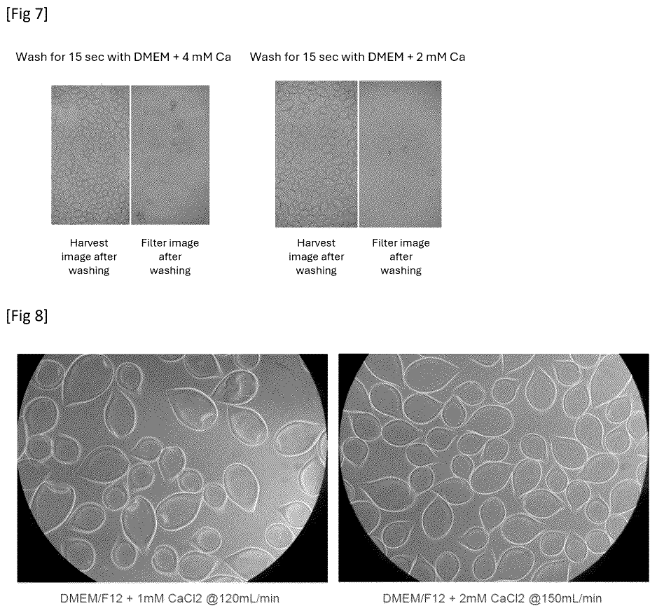

After the rinsing step, a photograph of the microcompartments recovered in DMEM/F-12 was taken by microscopy after rinsing (FIG. 7), along with a photograph of the remaining microcompartments trapped in the filter used at the end of rinsing. These photographs were obtained using a wide-field microscope at 2× magnification.

The observations show that the integrity of the microcompartments is preserved. A small proportion of cellular microcompartments were damaged when they crashed into the filter, demonstrating increased mechanical resistance.

Furthermore, no significant aggregation of microcompartments was observed on the filter, by virtue of the calcium supplementation in the rinsing solution. These results demonstrate that calcium (Ca²⁺) supplementation of the rinsing solution significantly improves the mechanical strength of crosslinked cellular microcompartments during rinsing, as shown with device 1.

### Example 3: Evaluating the Mechanical Strength of Microcompartments During Filtration at Different Rinse Flow Rates According to the Treatment Method of the Invention (Calcium)

A 3 mL volume of microcompartments was deposited in a reversible cell sieve with a porosity of 100 μm. The microcompartments were then rinsed with different solutions, either DMEM alone or DMEM supplemented with increasing concentrations of calcium, as listed in Table 1. Rinsing was carried out for 1 minute at different flow rates.

After this step, the microcompartments were examined under a phase contrast microscope at 4× magnification to assess any damage, notably ruptures (FIG. 8). It was observed that the microcompartments rinsed with DMEM alone were damaged as early as a rinse flow rate of 60 mL/min. In contrast, the addition of calcium to DMEM increased the rinse flow rate without damaging the microcompartments.

In detail, a concentration of 1 mM of CaCl₂ preserved the integrity of the microcompartments up to a flow rate of 120 mL/min, but damage was observed beyond this (FIG. 8). With 2 mM of CaCl₂ or more, the microcompartments remained intact even at higher flow rates, up to 180 mL/min.

Table 1 summarizes the microscopic observations, showing that microcompartments washed with calcium-supplemented DMEM benefit from improved mechanical strength. The optimum concentration observed is 2 mM of CaCl₂ or more, guaranteeing protection against damage at high flow rates.

TABLE 1

| | |
|---|---|
| Rinsing | Flow rate (mL/min) |

| medium | 30 | 60 | 90 | 120 | 150 | 180 |
|---|---|---|---|---|---|---|
| DMEM | − | + | + | + | + | + |
| DMEM + | − | − | − | + | + | + |
| 1 mM CaCl2 | | | | | | |
| DMEM + | − | − | − | − | − | − |
| 2 mM CaCl2 | | | | | | |
| DMEM + | − | − | − | − | − | − |
| 5 mM CaCl2 | | | | | | |
| 1 mM CaCl2 | − | − | − | − | − | − |
| 2 mM CaCl2 | − | − | − | − | − | − |
| 5 mM CaCl2 | − | − | − | − | − | − |
| 25 mM | − | − | − | − | − | − |
| CaCl2 | | | | | | |

The − symbol means that microscopic observation has failed to identify any damage to the microcompartments.

The + symbol means that microscopic observation has identified damage to the microcompartments.

### Example 4: Demonstrating the Effect of the Treatment Method According to the Invention (Iron) on the Mechanical Strength of the Microcompartments, in the Context of Filtering Using a Cell Sieve

This example shows an embodiment of the invention wherein the rinsing solution is supplemented with iron (FeCl₃). Three distinct cell lines were tested (cell line no. 1, 2 and 3) while being mixed with a cell culture medium.

Solutions were co-injected using a micro-fluidic injector comprising three lines upstream of the nozzle: a first line containing suspended cells, a second line containing a 2% alginate solution, and a third line containing an intermediate sorbitol solution. After encapsulation, the resulting drops were collected in a calcium bath (CaCl₂), allowing the alginate to crosslink and the formation of the cellular microcompartments.

After encapsulation, the microcompartments were rinsed with serum-free culture medium. A 1 mL volume of microcompartments was deposited in a reversible cell sieve (100 μm), then a solution of DMEM supplemented with FeCl₃ concentrations of 0.79 mM or 8.9 mM (75 mL) was injected to perform the rinsing step. The rinsed microcompartments were observed by microscopy.

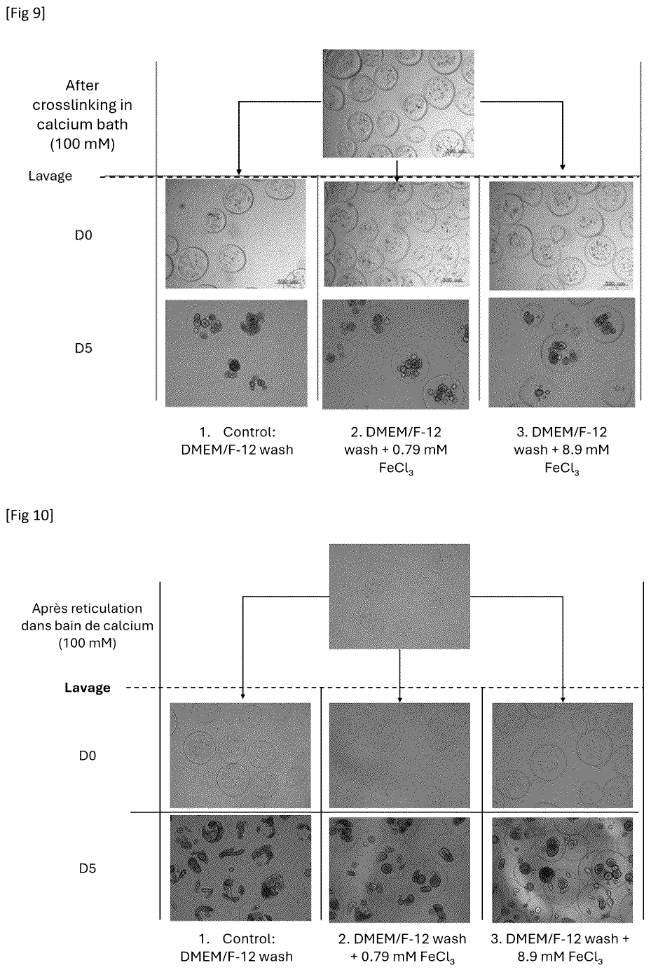

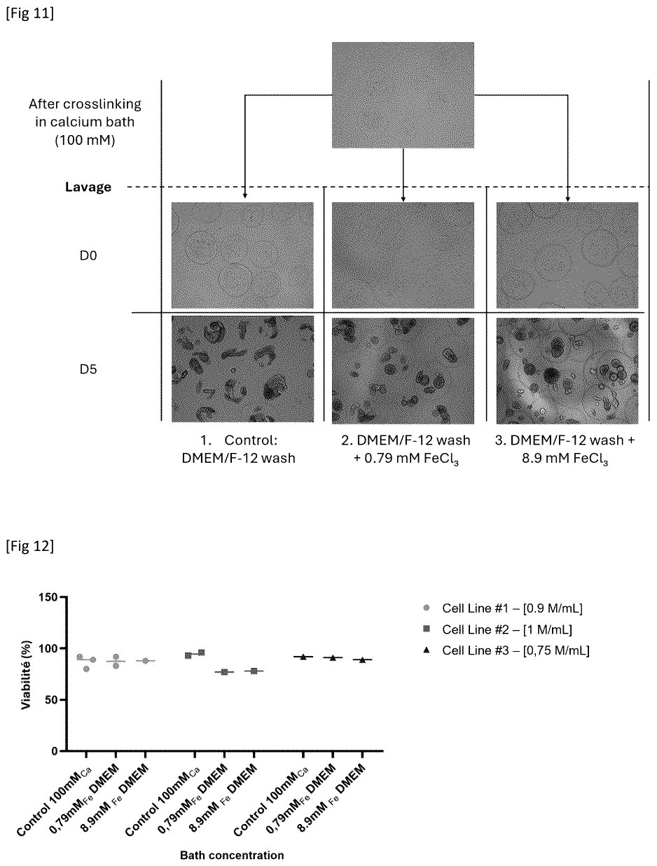

The results are shown in FIGS. 9 (cell line no. 1), 10 (cell line no. 2) and 11 (cell line no. 3).

In general, the cellular microcompartments have a stiffer and harder outer shell after rinsing with a solution comprising iron.

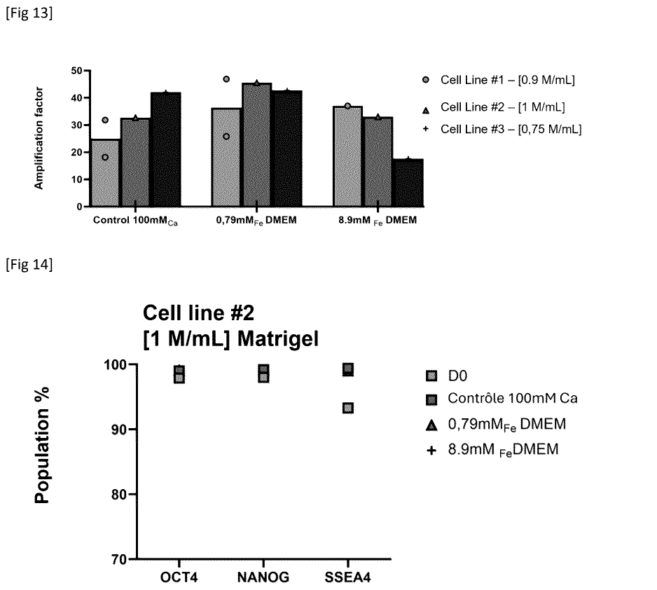

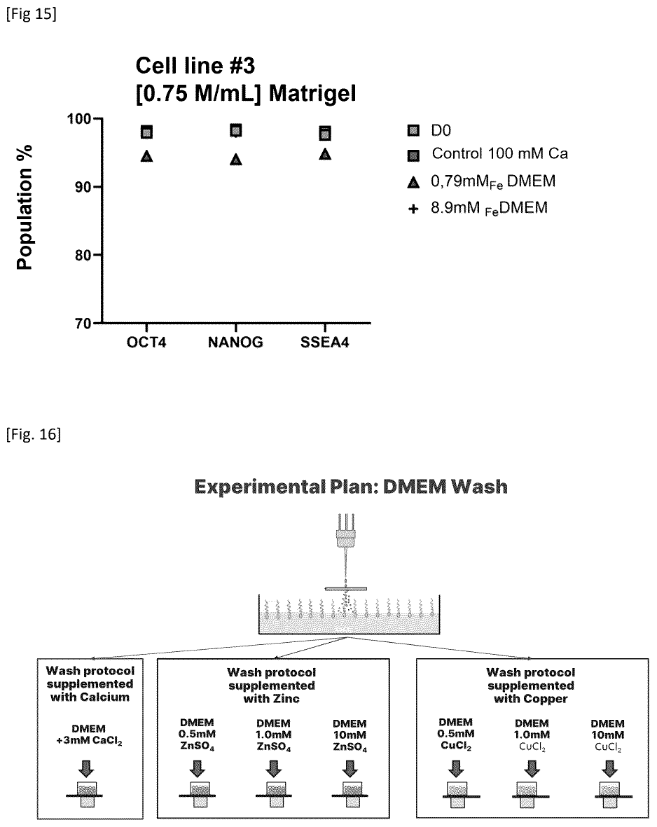

After 5 days of culture in a flask, the microcompartments were dissolved and the cells were dissociated to assess their viability (FIG. 12), their amplification factor (FIG. 13) and their pluripotency (FIGS. 14 and 15).

Cell lines no. 1 and no. 2 show greater amplification than the controls.

These results demonstrate an improved mechanical strength of the cellular microcompartments subjected to rinsing with a rinsing solution comprising iron, with no negative impact on their amplification, their viability or their ability to maintain their pluripotency.

### Example 5: Demonstrating the Effect of the Treatment Method According to the Invention (Zinc and Copper) on the Mechanical Strength of the Microcompartments and Cell Viability

This example shows one embodiment of the invention wherein the rinsing solution is supplemented with zinc (ZnSO₄) or copper (CuCl₂) at different concentrations.

Cellular microcompartments containing pluripotent stem cells were prepared by co-injecting solutions using a micro-fluidic injector, then crosslinked in a calcium bath (CaCl₂)).

The microcompartments were then rinsed with different DMEM solutions supplemented as follows:
3 mM CaCl₂ (control)
0.5 mM ZnSO₄
1 mM ZnSO₄
10 mM ZnSO₄
0.5 mM CuCl₂
1 mM CuCl₂
10 mM CuCl₂

The microcompartments were cultured for 5 days with daily renewal of the culture medium (1 mL.

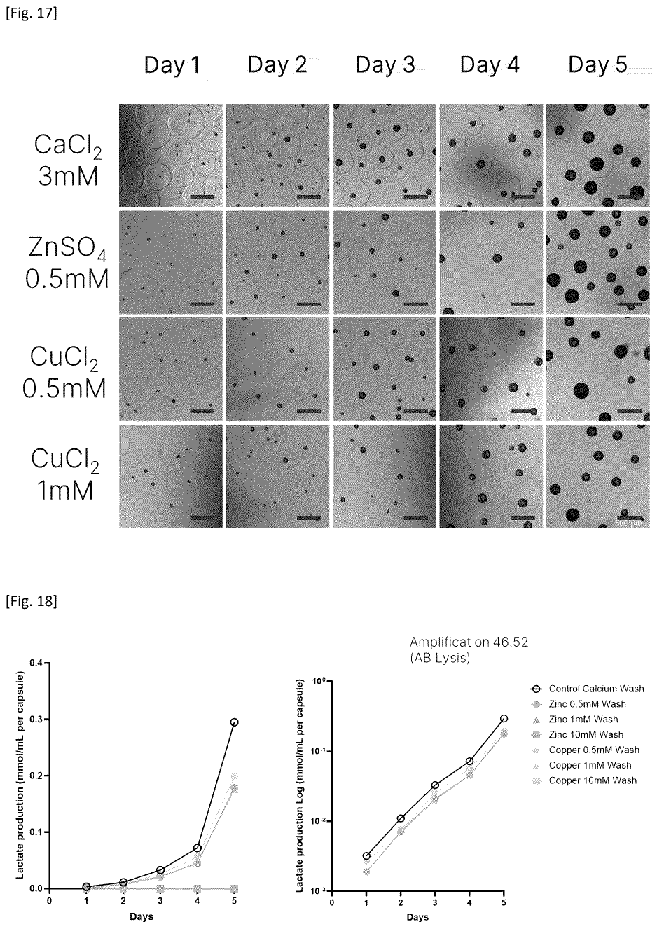

The results are shown in FIGS. 16 to 19.

The cultures that showed satisfactory cell growth were those rinsed with:
3 mM CaCl₂ (control)
0.5 mM ZnSO₄
0.5 mM CuCl₂
1 mM CuCl₂

In contrast, the cultures rinsed with 1 mM ZnSO₄, 10 mM ZnSO₄ and 10 mM CuCl₂ showed no cell growth, indicating that these concentrations are too high to maintain cell viability.

The cells grown in the microcompartments rinsed with 0.5 mM ZnSO₄, 0.5 mM CuCl₂ and 1 mM CuCl₂ show similar amplification and viability to those of the control (3 mM CaCl₂) after 5 days in culture.

Lactate production, an indicator of cellular metabolic activity, follows a similar trend for the 0.5 mM ZnSO₄, 0.5 mM CuCl₂ and 1 mM CuCl₂ conditions with respect to the 3 mM CaCl₂ control ([FIG. 18]).

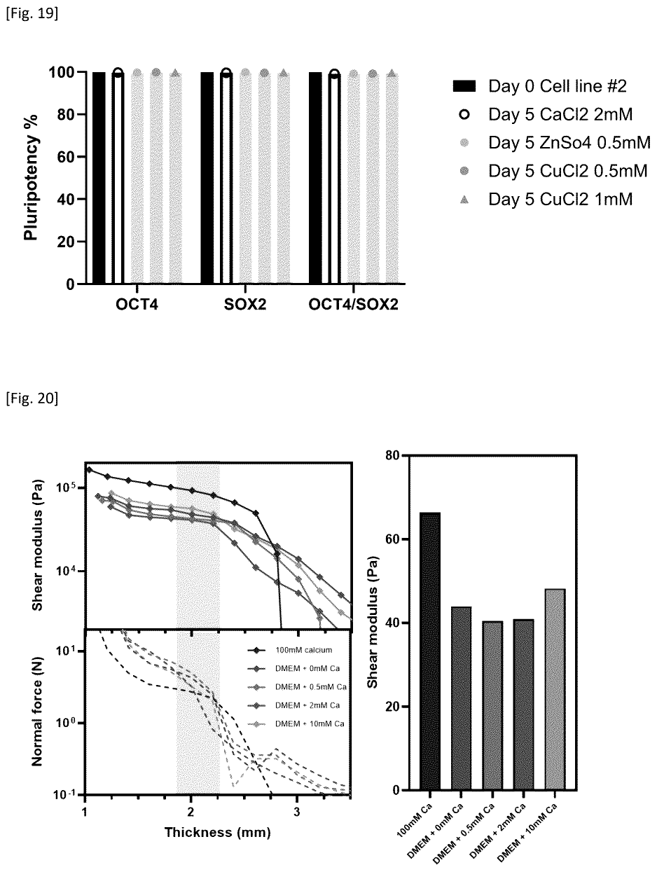

The pluripotency measurements show that the cells maintain a high expression of the OCT4, SOX2 and OCT4/SOX2 markers (close to 100%) for all the conditions that enabled cell growth ([FIG. 19]).

These results demonstrate that zinc at a concentration of 0.5 mM and copper at concentrations of 0.5 mM and 1 mM can be used as alternatives or complements to calcium in the rinsing solution, making it possible to reinforce the mechanical properties of the microcompartments while preserving the viability, proliferation capacity and pluripotency of the encapsulated cells.

### Example 6: Characterizing the Mechanical Properties of Alginate Hydrogels by Rheological Measurements

This example shows the characterization of the mechanical properties of bulk alginate hydrogels after different washing treatments.

Bulk hydrogel manufacturing protocol:

Bulk alginate gels were manufactured using a mold composed of two plastic parts separated by a dialysis membrane (10 kDa MWCO).

2 mL of liquid alginate was poured into the mold. The mold was immersed in 30 mL of a 100 mM calcium bath for 1.5 hours, then the bath was renewed and left for 3.5 hours.

The bath was then replaced with the washing medium for 30 minutes, renewed and left overnight, then renewed and left for 1 hour.

The washing media tested were as follows:
100 mM Ca (crosslinking control)
DMEM+0 mM Ca
DMEM+0.5 mM Ca
DMEM+2 mM Ca
DMEM+10 mM Ca

Rheological measurements: the gel was removed from the mold and placed in the rheometer geometry (25 mm diameter sanded plane/plane geometry). The geometry was lowered in 0.2 mm increments, the sample was left to equilibrate for each increment, then an oscillatory measurement was taken at 1 Hz and 0.1% strain.

The results are presented in the table below and shown in [FIG. 21]:

TABLE 2

| Condition | Shear modulus (kPa) | Young's modulus (kPa) |
|---|---|---|
| 100 mM Ca | 67 | 181 |
| DMEM + 0 mM Ca | 44 | 119 |
| DMEM + 0.5 mM Ca | 40 | 108 |
| DMEM + 2 mM Ca | 41 | 111 |
| DMEM + 10 mM Ca | 48 | 130 |

The Young's modulus E was calculated from the shear modulus G according to the relationship E=3G, assuming a Poisson's ratio v=035.

These results show that the concentration of calcium in the washing medium influences the mechanical properties of the hydrogel. Washing with a medium containing 10 mM of calcium maintains a higher shear modulus (48 kPa) with respect to washing without additional calcium (44 kPa) or with intermediate concentrations (40-41 kPa).

The highest shear modulus (67 kPa) is obtained for gels kept in the crosslinking bath with 100 mM of calcium, which corresponds to the crosslinking maximum of alginate.

These rheological measurements confirm that the rinsing treatment with a solution containing cations, notably calcium, modulates and reinforces the mechanical properties of alginate hydrogels.

1. A method for treating a three-dimensional microcompartment comprising at least one biological element encapsulated in a crosslinked hydrogel, so as to reinforce the mechanical properties of said microcompartment, characterized in that it comprises a step of rinsing the microcompartment in a rinsing solution comprising at least one cation.

2. The method according to claim 1, characterized in that the rinsing solution is a solution in which at least one cation has a concentration of between 0.5 and 50 mM; and wherein the at least one cation of the rinsing solution is selected from calcium, iron, barium, strontium, copper, lead, aluminum, magnesium, manganese, zinc, cobalt, nickel ions and combinations thereof.

3. (canceled)

4. The method according to claim 2, wherein the rinsing solution comprises one or more of the following:
calcium at a concentration of between 0.5 and 10 mM;
zinc at a concentration of between 0.1 and 5 mM;
zinc sulfate (ZnSO₄);
copper at a concentration of between 0.1 and 5 mM;
copper chloride (CuCl₂);
calcium at a concentration ranging from 0.5 to 10 mM and zinc at a concentration ranging from 0.1 to 1 mM; or
calcium at a concentration ranging from 0.5 to 10 mM and copper at a concentration ranging from 0.1 to 2 mM.

5. (canceled)

6. (canceled)

7. (canceled)

8. (canceled)

9. (canceled)

10. (canceled)

11. (canceled)

12. The method according to claim 1, characterized in that the microcompartment is hollow and in that it comprises an outer hydrogel layer defining an inner part comprising at least one biological element.

13. The method according to claim 12, characterized in that the three-dimensional microcompartment is hollow, and consists of an outer hydrogel layer encapsulating a hollow inner part comprising at least:
one or more biological elements, and
optionally culture medium and/or extracellular matrix elements.

14. The method according to claim 1, characterized in that the microcompartment is a solid hydrogel ball encapsulating at least one biological element; and wherein the hydrogel is selected from alginates, carrageenans, pectins, gelatins, collagen, hyaluronic acid, chitosan or any combination thereof.

15. (canceled)

16. (canceled)

17. The method according to claim 1, characterized in that at least one biological element encapsulated in the microcompartment is selected from: a cell, a cell organelle, a cell fragment, a cell aggregate, an organoid, DNA, RNA, a protein, a microorganism, a vector, a vesicle, an exosome, a microtissue, a spheroid, an embryoid body and combinations thereof.

18. The method according to claim 17, characterized in that at least one cell is selected from stem cells, progenitor cells, differentiated cells, cancer cells and combinations thereof.

19. The method according to claim 1, characterized in that the microcompartment encapsulates at least one human biological element.

20. The method according to claim 1, wherein the microcompartment is enclosed and is further characterized by one or more of the following features:
the microcompartment has the shape of an ovoid, a cylinder, a spheroid, a sphere, a fiber or a teardrop; and/or
the smallest dimension of the microcompartment is between 1 μm and 1.5 mm.

21. (canceled)

22. (canceled)

23. The method according to claim 1, characterized in that it comprises a step of mechanically stressing said rinsed microcompartment, wherein the mechanical stressing is performed by filtration, agitation in liquid medium, centrifugation, suction or a combination thereof.

24. The method according to claim 23, characterized in that the mechanical stress step applies a centrifugal acceleration of at least 10 g to at least one rinsed microcompartment.

25. The method according to claim 1, characterized in that the rinsing step and/or the mechanical stress step are carried out using at least one device comprising a body (2) having at least a first port (5) and a second port (6) and a filtering membrane (3) extending along a plane P in the body (2), thus defining two compartments and able to prevent the passage of cellular microcompartments from one compartment to the other, the first port (5) being intended for the injection and withdrawal of the cellular microcompartments immersed in a soaking solution and/or a rinsing solution and the second port (6) being intended for the withdrawal and injection of the soaking solution and/or the rinsing solution, characterized in that the injection into the first port (5) takes place in a direction D that is not substantially orthogonal to the plane P of the filtering membrane (3).

26. A method for preparing a three-dimensional microcompartment comprising the implementation of the following steps:
a) Encapsulating at least one biological element in a crosslinked hydrogel to form a three-dimensional microcompartment;
b) Treating the microcompartment obtained in step a) according to the method of claim 1.

27. The method according to claim 26, characterized in that encapsulation step a) comprises a step of crosslinking the hydrogel using a crosslinking solution comprising at least one crosslinking agent.

28. The method according to claim 27, characterized in that at least one crosslinking agent is calcium.

29. (canceled)

30. The method according to claim 26, characterized in that at least one crosslinking agent of step a) is distinct from at least one cation of step b).

31. The method according to claim 26, characterized in that at least one crosslinking agent of step a) is identical to at least one cation of step b).

32. The method according to claim 26, characterized in that step a) comprises the following steps:
1) forming a hydrogel microcompartment encapsulating biological elements, using an encapsulation device, preferentially comprising a micro-fluidic and/or milli-fluidic device, and solutions, said solutions preferentially being the following:
a hydrogel solution;
optionally an intermediate solution, preferentially isotonic; and
a solution comprising biological elements, culture medium and, optionally, the extracellular matrix and/or extracellular matrix substitute;
2) crosslinking the hydrogel, preferentially using a crosslinking solution.

33. The method according to claim 32, characterized in that step a) comprises the following steps
1) co-injecting two or three solutions to form a hollow hydrogel microcompartment encapsulating biological elements, said solutions being selected from:
a hydrogel solution;
optionally an intermediate solution, preferentially isotonic; and
a solution comprising biological elements, culture medium and optionally extracellular matrix and/or extracellular matrix substitute, concentrically via a micro-fluidic injector which forms a jet at the injector outlet consisting of the mixture of solutions, said jet breaking up into drops;
2) crosslinking the hydrogel.

34. The method according to claim 26, wherein the method further comprises culturing the encapsulated cells in the microcompartment, in a bioreactor and/or cryopreserving the obtained microcompartments.

35. (canceled)

36. (canceled)

37. A three-dimensional microcompartment obtained by a method according to claim 1, wherein the three-dimensional microcompartment is characterized by one or more of the following features:
a Young's modulus between 3 kPa and 1.2 MPa, preferentially between 3 and 800 kPa, notably between 3 and 600 kPa, between 3 and 500 kPa, preferentially between 3 and 350 kPa;
a rupture stress of between 10 and 500 kPa, notably between 200 and 500 kPa; and/or
a shear modulus ranging from 30 to 80 kPa.

38. (canceled)

39. (canceled)

40. (canceled)

41. (canceled)

42. (canceled)

43. (canceled)

44. (canceled)
